(19) United States
(12) Patent Application Publication
(10) Pub. No.: US 2018/0127800 A1
(43) Pub. Date: May 10, 2018

(54) COMPOSITIONS AND METHODS FOR IDENTIFYING ZIKA ENZYME INHIBITORS

(71) Applicant: San Diego State University (SDSU) Foundation, dba San Diego State University Research Foundation, San Diego, CA (US)

(72) Inventor: Roland Wolkowicz, San Diego, CA (US)

(21) Appl. No.: 15/791,040

(22) Filed: Oct. 23, 2017

Related U.S. Application Data
(60) Provisional application No. 62/411,661, filed on Oct. 23, 2016.

(51) Int. Cl. C12Q 1/37; C07K 5/11; C07K 14/435

(52) U.S. Cl. CPC C12Q 1/37; C07K 14/43504; C07K 5/1019

(57) ABSTRACT

In alternative embodiments, provided are compositions, e.g., multiplexed platforms or systems, to screen for small molecule drugs that inhibit enzymes such as proteases, e.g., viral proteases, including Zika Virus (ZIKV). In one embodiment, provided are cell-based or multiplexed platforms for monitoring the activity of enzymes, e.g., proteases such as Zika Virus (ZIKV) viral proteases. In alternative embodiments, provided are assays that monitor the activity of the viral ZIKV NS3 protease, as observed by the cleavage of its boundaries with its cofactor NS2B and its downstream nonstructural protein NS4A.

### RELATED APPLICATIONS

This U.S. utility patent application claims the benefit of priority under 35 U.S.C. § 119(e) of U.S. Provisional Application No. 62/411,661, filed Oct. 23, 2016. The aforementioned application is expressly incorporated herein by reference in its entirety and for all purposes.

### TECHNICAL FIELD

This invention relates to molecular and cellular biology, biochemistry, molecular genetics, and drug design and discovery. In alternative embodiments, provided are compositions, e.g., multiplexed platforms or systems, to screen for small molecule drugs that inhibit enzymes such as proteases, e.g., viral proteases, including Zika Virus (ZIKV). In one embodiment, provided are cell-based or multiplexed platforms for monitoring the activity of enzymes, e.g., proteases such as Zika Virus (ZIKV) viral proteases. In alternative embodiments, provided are assays that monitor the activity of the viral ZIKV NS3 protease, as observed by the cleavage of its boundaries with its cofactor NS2B and its downstream nonstructural protein NS4A.

### BACKGROUND

The Zika Virus (ZIKV) has gained the spotlight and the interest of the research community due to its ability to impair human development. Microcephaly in newborn infants has raised the concerns of World Health Organizations. The World Health Organization (WHO) declared the ZIKV epidemic a global health emergency on Feb. 1, 2016. There is currently no treatment or vaccine available for ZIKV.

ZIKV is an arthropod-borne virus (arbovirus) grouped into the Flavivirus genus within the viral Flaviviridae family. Among the Flavivirus genus Zika is accompanied by other well-known pathogens that infect both humans and animals such as Dengue Virus (DenV), West Nile Virus (WNV), Yellow Fever Virus (YFV), and Japanese Encephalitis Virus (JEV). ZIKV is a prototypical Flavivirus characterized by its single stranded positive sense RNA genome enveloped by a small diameter capsid/envelope structure. The genome codes for single polyprotein that contains three structural proteins: capsid (C), envelope (E), and membrane (M) as well as seven nonstructural proteins: NS1, NS2A, NS2B, NS3, NS4A, NS4B, and NS5. The genome contains a 5′ Cap like structure and is directly translated by the host ribosomes spanning the endoplasmic reticulum (ER) in such a way that segments are embedded within the lumen of the ER while others remain in the cytosol due to the hydrophobic regions of the polyprotein, the topology of which is well documented.

ZIKV and Flaviviruses alike depend on proteolytic cleavage of the polyprotein for replication to occur. Cleavage is carried out by both the viral serine protease NS3 together with its co-factor NS2B, and by the host protease Furin (or similar) in a site-specific manner across the viral proteome. The autocatalytic in-cis cleavage of NS3 out of the polypeptide is required for the in-trans cleavage and processing of the remaining proteome. Thus, in the presence of a protease inhibitor (PI) autocatalytic cleavage would be blocked along with the continuing replication of the virus. This type of cleavage was successfully monitored by a previously developed assay for HIV-1. The assay was developed in mammalian cells and relied on an engineered Gal4/protease fusion protein that travels to the nucleus and activates the GFP reporter gene when inhibited. The activation or lack thereof of the reporter GFP gene can be measured and quantified via flow cytometry.

ZIKV life cycle is directly dependent upon the proteolytic processing of its viral proteome into its corresponding proteins. Inhibition of this cleavage by blocking the first step of autocatalytic cleavage of NS3 will absolutely disrupt the life cycle of the virus rendering future replication impossible.

### SUMMARY

In alternative embodiments, provided are cell-based methods, cell-based platforms or systems, or multiplexed platforms or systems, for monitoring the activity of a Zika Virus (ZIKV) protease, e.g., a ZIKV NS3 protease, comprising (or made by a method comprising):

(1) (a) providing a nucleic acid encoding a scaffold protein (or one or more scaffold proteins) operatively linked to a transcriptional regulatory unit, wherein the scaffold protein comprises:
(i) an amino acid motif or subsequence susceptible to cleavage by a Zika Virus (ZIKV) protease or ZIKV NS3 protease, or equivalent, under physiologic (cell culture) conditions;
(ii) a transmembrane domain;
(iii) a signal sequence or any amino acid motif that places the scaffold protein or proteins on the extracellular surface of the cell; and
(iv) a detectable moiety,
wherein the amino acid motif or subsequence susceptible to cleavage by the ZIKV protease or equivalent is positioned within the scaffold protein such that when the detectable moiety is cleaved away from (off from) the scaffold protein by the ZIKV protease or equivalent the remaining subsequence of scaffold protein on the extracellular surface of the cell lacks the detectable moiety;

(b) providing a nucleic acid encoding the ZIKV protease or equivalent operatively linked to a transcriptional regulatory unit, or a cell that expresses a heterologous or endogenous ZIKV protease or equivalent,

and optionally the ZIKV protease or equivalent further comprises the ZIKV protease NS2B cofactor, i.e., the nucleic acid encodes both the ZIKV protease or equivalent and the ZIKV protease NS2B cofactor, or provided is one nucleic acid encoding the ZIKV protease or equivalent and one nucleic acid encoding the ZIKV protease NS2B cofactor;

(c) inserting (transfecting) the nucleic acid(s) of (a) and (b) into the cell if the cell does not already express the heterologous or endogenous ZIKV protease or equivalent;

(d) co-expressing the nucleic acid of (a) and (b) in the cell, or expressing the nucleic acid of (a) in the cell if the cell already expresses a heterologous or endogenous ZIKV protease or equivalent and/or a ZIKV protease NS2B cofactor; and

(e) determining whether the scaffold protein comprising the detectable moiety is expressed on the extracellular surface of the cell,

wherein an intact scaffold protein comprising the detectable moiety is expressed on the extracellular surface of the cell when the ZIKV protease or equivalent is not enzymatically active, and an intact scaffold protein is not or is substantially less expressed on the extracellular surface of the cell when the ZIKV protease or equivalent is enzymatically active (the detectable moiety is cleaved off by the ZIKV protease or equivalent);

(2) the cell-based method, cell-based platform or system or multiplexed platform of (1), wherein the scaffold protein further comprises an endoplasmic reticulum (ER) retention motif or a KDEL (SEQ ID NO:1) motif,

wherein the ER retention motif or KDEL (SEQ ID NO:1) motif is positioned in the scaffold protein such that when the ZIKV protease or equivalent is active the scaffold will be separated into two pieces, leaving the ER retention motif-comprising or KDEL (SEQ ID NO:1) motif-comprising portion of the polypeptide in the ER and freeing the detectable moiety-comprising portion to the cell's extracellular membrane, and if the ZIKV protease or equivalent is blocked or inactive, the entire scaffold polypeptide will be retained in the ER, and as a consequence will not be detected on the cell's extracellular surface;

(3) the cell-based method, cell-based platform or system or multiplexed platform of any of (1) to (2), further comprising screening for an inhibitor of the ZIKV protease or equivalent by:
(a) providing a compound to be screened as an inhibitor of the ZIKV protease or equivalent, or providing a nucleic acid to be screened as encoding an inhibitor of the ZIKV protease or equivalent;
(b) contacting a plurality of the cells with the compound or nucleic acid of (a) either before, during and/or after the co-expressing the nucleic acid in the cell; and
(c) determining whether the scaffold protein comprising the detectable moiety is expressed on the extracellular surface of the cell,
wherein an intact scaffold protein comprising the detectable moiety is expressed on the extracellular surface of the cell when the ZIKV protease or equivalent is inhibited by: the compound, a composition encoded by the nucleic acid, or a compound present in the cell only because the nucleic acid was expressed,
and an intact scaffold protein is not or is substantially less expressed on the extracellular surface of the cell when the ZIKV protease or equivalent is enzymatically active (the detectable moiety is cleaved off by the ZIKV protease or equivalent) and the enzymatic activity of the ZIKV protease or equivalent is not significantly inhibited by: the compound, a composition encoded by the nucleic acid, or a compound present in the cell only because the nucleic acid was expressed;

(5) the cell-based method, cell-based platform or system or multiplexed platform of any of (1) to (4), further comprising running a negative control comprising dividing the plurality of the cells co-expressing the nucleic acid of (a) and (b) in the cell and not adding the compound to be screened as an inhibitor to one of the divided cell samples;

(6) the cell-based method, cell-based platform or system or multiplexed platform of any of (1) to (5), further comprising running a positive control comprising dividing the plurality of the cells co-expressing the nucleic acid of (a) and (b) in the cell and adding a known inhibitor of the ZIKV protease or equivalent to one of the divided cell samples;

(7) the cell-based method, cell-based platform or system or multiplexed platform of any of (1) to (6), wherein the amino acid motif or subsequence susceptible to cleavage by the ZIKV protease or equivalent under physiologic (cell culture) conditions comprises:
the six amino acids upstream of NS2B (at the boundary of NS2A),
the amino acids downstream of NS3 (boundary with NS4A), or
the six amino acids upstream of NS2B (at the boundary of NS2A) and the amino acids downstream of NS3 (boundary with NS4A),
and optionally the two sets of two hydrophobic transmembrane domains (TMs) that anchor wild type NS2B to the ER are deleted;

(8) the cell-based method, cell-based platform or system or multiplexed platform of any of (1) to (7), wherein:
the ZIKV NS3 proteases have amino acid sequences: ZIKV-2013 (SEQ ID NO:6); ZIKV-2007 (SEQ ID NO:7); ZIKV-1947 (SEQ ID NO:8);
the ZIKV NS2B co-factors have amino acid sequences: ZIKV-2013 (SEQ ID NO:2); ZIKV-2007 (SEQ ID NO:3); ZIKV-1947 (SEQ ID NO:4);

(9) the cell-based method, cell-based platform or system or multiplexed platform of any of (1) to (8), wherein the transcriptional regulatory unit comprises a promoter, an inducible promoter or a constitutive promoter;

(10) the cell-based method, cell-based platform or system or multiplexed platform of any of (1) to (9), wherein the cell is a mammalian cell, a monkey cell or a human cell, or a lymphocyte or a hepatocyte, or a T cell, and optionally the cells are genetically bar-coded;

(11) the cell-based method, cell-based platform or system or multiplexed platform of any of (1) to (10), wherein the scaffold proteins comprise all or part of a mouse Lyt2 or a human CD8 polypeptide;

(12) the cell-based method, cell-based platform or system or multiplexed platform of any of (1) to (11), wherein the detectable moiety comprises an epitope for an antibody, or a FLAG tag;

(13) the cell-based method, cell-based platform or system or multiplexed platform of any of (1) to (12), wherein the detectable moiety is detected or measured on the extracellular surface of the cell by a high throughput screen, a plate-reader, a flow cytometry or microscope visualization;

(14) the cell-based method, cell-based platform or system or multiplexed platform of any of (1) to (13), wherein the compound to be screened as an inhibitor of the ZIKV protease comprises a small molecule, a nucleic acid, a polypeptide or peptide, a peptidomimetic, a polysaccharide or a lipid;

(15) the cell-based method, cell-based platform or system or multiplexed platform of any of (1) to (14), wherein the compound to be screened as an inhibitor of the ZIKV protease is a member of a library of compounds to be screened, or a member of a random peptide library or a chemical compound library; or

(16) the cell-based method, cell-based platform or system or multiplexed platform of any of (1) to (15), wherein two or more, or a plurality of, ZIKV protease are screened in the same cell, wherein optionally they are variants of the same ZIKV protease, or a combination thereof.

In alternative embodiments, provided are cell-based platforms, multiplexed platforms or systems, or cell-based methods, for monitoring the activity of an ZIKV protease comprising (or made by a method comprising):

(1) (a) providing a nucleic acid encoding a scaffold protein (or one or more scaffold proteins) operatively linked to a transcriptional regulatory unit, wherein the scaffold protein comprises:
(i) an amino acid motif or subsequence susceptible to cleavage by the ZIKV protease or equivalent under physiologic (cell culture) conditions;
(ii) a transmembrane domain;
(iii) a signal sequence or any amino acid motif that places the scaffold protein or proteins on the extracellular surface of the cell; and
(iv) a detectable moiety,
wherein the amino acid motif or subsequence susceptible to cleavage by the ZIKV protease is positioned within the scaffold protein such that when the detectable moiety is cleavage away from (off from) the scaffold protein by the ZIKV protease the remaining subsequence of scaffold protein on the extracellular surface of the cell lacks the detectable moiety;

(b) providing a nucleic acid encoding the ZIKV protease operatively linked to a transcriptional regulatory unit, or a cell that expresses a heterologous or endogenous ZIKV protease;

(c) inserting (transfecting) the nucleic acid of (a) and (b) into the cell if the cell does not already express a heterologous or endogenous ZIKV protease or equivalent;

(d) co-expressing the nucleic acid of (a) and (b) in the cell, or expressing the nucleic acid of (a) in the cell if the cell already expresses a heterologous or endogenous ZIKV protease or equivalent and/or a ZIKV protease NS2B cofactor; and

(e) determining whether the scaffold protein comprising the detectable moiety is expressed on the extracellular surface of the cell,

wherein an intact scaffold protein comprising the detectable moiety is expressed on the extracellular surface of the cell when the ZIKV protease or equivalent is not enzymatically active, and an intact scaffold protein is not or is substantially less expressed on the extracellular surface of the cell when the ZIKV protease or equivalent is enzymatically active (the detectable moiety is cleaved off by the ZIKV protease or equivalent); or

(2) the cell-based platform, multiplexed platform or system, or cell-based method of (1), wherein the scaffold protein further comprises an endoplasmic reticulum (ER) retention motif or a KDEL (SEQ ID NO:1) motif,

wherein the ER retention motif or KDEL (SEQ ID NO:1) motif is positioned in the scaffold protein such that when ZIKV protease or equivalent is active the scaffold will be separated into two pieces, leaving the ER retention motif-comprising or KDEL (SEQ ID NO:1) motif-comprising portion of the polypeptide in the ER and freeing the detectable moiety-comprising portion to the cell's extracellular membrane, and if ZIKV protease or equivalent is blocked or inactive, the entire scaffold polypeptide will be retained in the ER, and as a consequence will not be detected on the cell's extracellular surface;

(3) the cell-based platform, multiplexed platform or system, or cell-based method of (1) or (2), further comprising screening for an inhibitor of ZIKV protease or equivalent by:
(a) providing a compound to be screened as an inhibitor of ZIKV protease or equivalent, or providing a nucleic acid to be screened as encoding an inhibitor of ZIKV protease or equivalent;
(b) contacting a plurality of the cells with the compound or nucleic acid either before, during and/or after the co-expressing the nucleic acid in the cell; and
(c) determining whether the scaffold protein comprising the detectable moiety is expressed on the extracellular surface of the cell,
wherein an intact scaffold protein comprising the detectable moiety is expressed on the extracellular surface of the cell when the ZIKV protease or equivalent is inhibited by: the compound, a composition encoded by the nucleic acid, or a compound present in the cell only because the nucleic acid was expressed, and an intact scaffold protein is not or is substantially less expressed on the extracellular surface of the cell when the ZIKV protease or equivalent is enzymatically active (the detectable moiety is cleaved off by the ZIKV protease or equivalent) and the enzymatic activity of the ZIKV protease or equivalent is not significantly inhibited by: the compound, a composition encoded by the nucleic acid, or a compound present in the cell only because the nucleic acid was expressed;

(4) the cell-based platform, multiplexed platform or system, or cell-based method of any of (1) to (3), further comprising running a negative control comprising dividing the plurality of the cells co-expressing the nucleic acid of (a) and (b) in the cell and not adding the compound to be screened as an inhibitor to one of the divided cell samples;

(5) the cell-based platform, multiplexed platform or system, or cell-based method of any of (1) to (4), further comprising running a positive control comprising dividing the plurality of the cells co-expressing the nucleic acid of (a) and (b) in the cell and adding a known inhibitor of the ZIKV protease or equivalent to one of the divided cell samples;

(6) the cell-based platform, multiplexed platform or system, or cell-based method of any of (1) to (5), wherein the transcriptional regulatory unit comprises a promoter;

(7) the cell-based platform, multiplexed platform or system, or cell-based method of any of (1) to (6), wherein the transcriptional regulatory unit comprises an inducible promoter or a constitutive promoter;

(8) the cell-based platform, multiplexed platform or system, or cell-based method of any of (1) to (7), wherein the cell is a mammalian cell, a monkey cell or a human cell, or a lymphocyte or a hepatocyte, or a T cell, and optionally the cells are genetically bar-coded;

(9) the cell-based platform, multiplexed platform or system, or cell-based method of any of (1) to (8), wherein the scaffold proteins comprise all or part of a mouse Lyt2 or a human CD8 polypeptide;

(10) the cell-based platform, multiplexed platform or system, or cell-based method of any of (1) to (9), wherein the detectable moiety comprises an epitope for an antibody, or a FLAG tag;

(11) the cell-based platform, multiplexed platform or system, or cell-based method of (10), wherein the detectable moiety is detected or measured on the extracellular surface of the cell by a high throughput screen, a plate reader, a flow cytometry or a microscope visualization;

(12) the cell-based platform, multiplexed platform or system, or cell-based method of any of (1) to (11), wherein the compound to be screened as an inhibitor of protease comprises a small molecule, a nucleic acid, a polypeptide or peptide, a peptidomimetic, a polysaccharide or a lipid, or, wherein the compound to be screened as an inhibitor of protease is a member of a library of compounds to be screened, or a member of a random peptide library or a chemical compound library; or

(13) the cell-based method, cell-based platform or multiplexed platform or system of any of (1) to (12), wherein two or more, or a plurality of, ZIKV protease or equivalent are screened in the same cell, wherein optionally they are variants of the same ZIKV protease or equivalent, or a combination thereof.

In alternative embodiments, provided are isolated, recombinant or synthetic nucleic acids encoding a scaffold protein (or one or more scaffold proteins) operatively linked to a transcriptional regulatory unit, wherein the scaffold protein comprises:

(1) (a) (i) an amino acid motif or subsequence susceptible to cleavage by a ZIKV protease or equivalent under physiologic (cell culture) conditions;
(ii) a transmembrane domain;
(iii) a signal sequence or any amino acid motif that places the scaffold protein on the extracellular surface of the cell; and
(iv) a detectable moiety; or

(b) the nucleic acid of (a), wherein the scaffold protein further comprises an endoplasmic reticulum (ER) retention motif or a KDEL (SEQ ID NO:1) motif,

wherein the ER retention motif or KDEL (SEQ ID NO:1) motif is positioned in the scaffold protein such that when a ZIKV protease or equivalent is active the scaffold will be separated into two pieces, leaving the ER retention motif-comprising or KDEL (SEQ ID NO:1) motif-comprising portion of the polypeptide in the ER and freeing the detectable moiety-comprising portion to the cell's extracellular membrane, and if the ZIKV protease or equivalent is blocked or inactive, the entire scaffold polypeptide will be retained in the ER, and as a consequence will not be detected on the cell's extracellular surface;

(2) the isolated, recombinant or synthetic nucleic acid of (1), wherein the ZIKV protease or equivalent further comprises its NS2B cofactor;

(3) the isolated, recombinant or synthetic nucleic acid of (1) or (2), wherein the scaffold protein comprise all or part of a mouse Lyt2 or a human CD8 polypeptide; or

(4) the isolated, recombinant or synthetic nucleic acid of (1), (2) or (3), wherein the detectable moiety comprises an epitope for an antibody, or a FLAG tag.

In alternative embodiments, provided are vectors, expression cassettes, cosmids or plasmids comprising (or having contained therein) the isolated, recombinant or synthetic nucleic acid as provided herein.

In alternative embodiments, provided are isolated, recombinant or synthetic polypeptides encoded by the nucleic acid as provided herein.

In alternative embodiments, provided are cells comprising (or having contained therein) an isolated, recombinant or synthetic nucleic acid provided herein, or a vector, expression cassette, cosmid or plasmid as provided herein, or a polypeptide as provided herein.

In alternative embodiments, provided are chimeric polypeptides comprising:
(1) (i) an amino acid motif or subsequence susceptible to cleavage by a ZIKV protease or equivalent (with or without its NS2B cofactor), under physiologic (cell culture) conditions;
(ii) a transmembrane domain;
(iii) a signal sequence or any amino acid motif that places the scaffold protein on the extracellular surface of the cell; and
(iv) a detectable moiety,

wherein the amino acid motif or subsequence susceptible to cleavage by the ZIKV protease or equivalent, is positioned within the scaffold protein such that when the detectable moiety is cleavage away from (off from) the scaffold protein by the ZIKV protease or equivalent, the remaining subsequence of scaffold protein on the extracellular surface of the cell lacks the detectable moiety;

(2) the chimeric polypeptide of (1), wherein the scaffold protein further comprises an endoplasmic reticulum (ER) retention motif or a KDEL (SEQ ID NO:1) motif,

wherein the ER retention motif or KDEL (SEQ ID NO:1) motif is positioned in the scaffold protein such that when the ZIKV protease or equivalent is active the scaffold will be separated into two pieces, leaving the ER retention motif-comprising or KDEL (SEQ ID NO:1) motif-comprising portion of the polypeptide in the ER and freeing the detectable moiety-comprising portion to the cell's extracellular membrane, and if the ZIKV protease or equivalent is blocked or inactive, the entire scaffold polypeptide will be retained in the ER, and as a consequence will not be detected on the cell's extracellular surface; or

(3) the chimeric polypeptide of (1) or (2), wherein the ZIKV protease or equivalent further comprises its NS2B cofactor.

In alternative embodiments, provided herein are chimeric or synthetic polypeptides comprising:
(1) (i) an amino acid motif or subsequence susceptible to cleavage by a ZIKV protease or equivalent under physiologic (cell culture) conditions;
(ii) a transmembrane domain;
(iii) a signal sequence or any amino acid motif that places the scaffold protein on the extracellular surface of the cell; and
(iv) a detectable moiety,

wherein the amino acid motif or subsequence susceptible to cleavage by the ZIKV protease or equivalent is positioned within the scaffold protein such that when the detectable moiety is cleavage away from (off from) the scaffold protein by the ZIKV protease or equivalent the remaining subsequence of scaffold protein on the extracellular surface of the cell lacks the detectable moiety;

(2) the chimeric polypeptide of (1), wherein the scaffold protein further comprises an endoplasmic reticulum (ER) retention motif or a KDEL (SEQ ID NO:1) motif,

wherein the ER retention motif or KDEL (SEQ ID NO:1) motif is positioned in the scaffold protein such that when ZIKV protease or equivalent is active the scaffold will be separated into two pieces, leaving the ER retention motif-comprising or KDEL (SEQ ID NO:1) motif-comprising portion of the polypeptide in the ER and freeing the detectable moiety-comprising portion to the cell's extracellular membrane, and if the ZIKV protease or equivalent e is blocked or inactive, the entire scaffold polypeptide will be retained in the ER, and as a consequence will not be detected on the cell's extracellular surface or

(3) the chimeric polypeptide of (1) or (2), wherein the ZIKV protease or equivalent further comprises its NS2B cofactor.

In alternative embodiments, provided herein are cell-based platforms, multiplexed platforms or systems, or cell-based methods, for monitoring the activity of a ZIKV protease or equivalent, comprising:

(1) (a) providing a nucleic acid encoding a scaffold protein (or one or more scaffold proteins) operatively linked to a transcriptional regulatory unit, wherein the scaffold protein comprises:
(i) an amino acid motif or subsequence susceptible to cleavage by the ZIKV protease or equivalent, under physiologic (cell culture) conditions;
(ii) a transmembrane domain;
(iii) a signal sequence or any amino acid motif that places the scaffold protein on the extracellular surface of the cell; and
(iv) a detectable moiety, a luminescent moiety, a Green Fluorescent Protein (GFP) or a luciferase, or any compound that can be directly or indirectly detected,
wherein the amino acid motif or subsequence susceptible to cleavage by the ZIKV protease or equivalent, is positioned within the scaffold protein such that when the detectable moiety is cleaved away from (off from) the scaffold protein by the ZIKV protease or equivalent the remaining subsequence of scaffold protein on the extracellular surface of the cell lacks the detectable moiety;

(b) providing a nucleic acid encoding a ZIKV protease or equivalent operatively linked to a transcriptional regulatory unit, or a cell that expresses a heterologous or endogenous ZIKV protease or equivalent;

(c) inserting (transfecting) the nucleic acid of (a) and (b) into the cell if the cell does not already express a heterologous or endogenous ZIKV protease or equivalent;

(d) co-expressing the nucleic acid of (a) and (b) in the cell, or expressing the nucleic acid of (a) in the cell if the cell already expresses a heterologous or endogenous ZIKV protease or equivalent; and

(e) determining whether the scaffold protein comprising the detectable moiety is expressed on the extracellular surface of the cell, optionally determined by a plate reader, flow cytometry or any high-throughput assay),

wherein an intact scaffold protein comprising the detectable moiety is expressed on the extracellular surface of the cell when the ZIKV protease or equivalent is not enzymatically active, and an intact scaffold protein is not or is substantially less expressed on the extracellular surface of the cell when the ZIKV protease or equivalent is enzymatically active (the detectable moiety is cleaved off by ZIKV protease or equivalent;

(2) the cell-based platform, multiplexed platform or system, or cell-based method of (1), wherein the scaffold protein further comprises an endoplasmic reticulum (ER) retention motif or a KDEL motif,

wherein the ER retention motif or KDEL motif is positioned in the scaffold protein such that when the ZIKV protease or equivalent is active the scaffold will be separated into two pieces, leaving the ER retention motif-comprising or KDEL motif-comprising portion of the polypeptide in the ER and freeing the detectable moiety-comprising portion to the cell's extracellular membrane, and if the ZIKV protease or equivalent is blocked or inactive, the entire scaffold polypeptide will be retained in the ER, and as a consequence will not be detected on the cell's extracellular surface;

(3) the cell-based platform, multiplexed platform or system, or cell-based method of (1), (2) or (3), further comprising screening for an inhibitor of the ZIKV protease or equivalent, by:
(a) providing a compound to be screened as an inhibitor of the ZIKV protease or equivalent, or providing a nucleic acid to be screened as encoding an inhibitor of the ZIKV protease or equivalent;
(b) contacting a plurality of the cells with the compound or nucleic acid of (a) either before, during and/or after the co-expressing the nucleic acid in the cell; and
(c) determining whether the scaffold protein comprising the detectable moiety is expressed on the extracellular surface of the cell,
wherein an intact scaffold protein comprising the detectable moiety is expressed on the extracellular surface of the cell when the ZIKV protease or equivalent is inhibited by: the compound, a composition encoded by the nucleic acid, or a compound present in the cell only because the nucleic acid was expressed, and an intact scaffold protein is not or is substantially less expressed on the extracellular surface of the cell when the ZIKV protease or equivalent is enzymatically active (the detectable moiety is cleaved off by the protease or enzyme, or the ZIKV protease or equivalent, and the enzymatic activity of the ZIKV protease or equivalent is not significantly inhibited by: the compound, a composition encoded by the nucleic acid, or a compound present in the cell only because the nucleic acid was expressed; or

(4) the cell-based platform, multiplexed platform or system, or cell-based method of any of (1) to (3), further comprising:
(a) running a negative control comprising dividing the plurality of the cells co-expressing the nucleic acid of (a) and (b) in the cell and not adding the compound to be screened as an inhibitor to one of the divided cell samples; or
(b) further comprising running a positive control comprising dividing the plurality of the cells co-expressing the nucleic acid of (a) and (b) in the cell and adding a known inhibitor of the ZIKV protease or equivalent to one of the divided cell samples;

(5) the cell-based platform, multiplexed platform or system, or cell-based method of any of (1) to (4), wherein:
(a) the amino acid motif or subsequence susceptible to cleavage by the ZIKV protease or equivalent under physiologic (cell culture) conditions comprises

the six amino acids upstream of NS2B (at the boundary of NS2A),

the amino acids downstream of NS3 (boundary with NS4A), or

the six amino acids upstream of NS2B (at the boundary of NS2A) and the amino acids downstream of NS3 (boundary with NS4A),

and optionally the two sets of two hydrophobic transmembrane domains (TMs) that anchor wild type NS2B to the ER are deleted;
(b)
   the ZIKV NS3 proteases have amino acid sequences: ZIKV-2013 (SEQ ID NO:6); ZIKV-2007 (SEQ ID NO:7); ZIKV-1947 (SEQ ID NO:8);
   the ZIKV NS2B co-factors have amino acid sequences: ZIKV-2013 (SEQ ID NO:2); ZIKV-2007 (SEQ ID NO:3); ZIKV-1947 (SEQ ID NO:4);
(c) the transcriptional regulatory unit comprises a promoter, an inducible promoter or a constitutive promoter;
(d) the cell is a mammalian cell, a monkey cell or a human cell, or a lymphocyte or a hepatocyte, or a T cell, and optionally the cells are genetically bar-coded;
(e) the scaffold proteins comprise all or part of a mouse Lyt2 or a human CD8 polypeptide;
(f) the detectable moiety comprises an epitope for an antibody, or a FLAG tag;
(g) the detectable moiety is detected or measured on the extracellular surface of the cell by a high throughput screen, a plate reader, a flow cytometry or a microscope visualization;
(h) the compound to be screened as an inhibitor of the ZIKV protease or equivalent comprises a small molecule, a nucleic acid, a polypeptide or peptide, a peptidomimetic, a polysaccharide or a lipid; or
(i) the compound to be screened as an inhibitor of the ZIKV protease or equivalent is a member of a library of compounds to be screened, or a member of a random peptide library or a chemical compound library; or

(6) the cell-based platform, multiplexed platform or system, or cell-based method of any of (1) to (5), wherein two or more, or a plurality of, ZIKV proteases or equivalents are screened in the same cell, wherein optionally they are variants of the same ZIKV protease or equivalent, or a combination thereof.

In alternative embodiments, provided herein are cell-based platforms, multiplexed platforms or systems, or cell-based methods, for monitoring the activity of a ZIKV protease or equivalent comprising:

(1) (a) providing a nucleic acid encoding a scaffold protein (or one or more scaffold proteins) operatively linked to a transcriptional regulatory unit, wherein the scaffold protein comprises:
(i) an amino acid motif or subsequence susceptible to cleavage by the ZIKV protease or equivalent under physiologic (cell culture) conditions;
(ii) a transmembrane domain;
(iii) a signal sequence or any amino acid motif that places the scaffold protein on the extracellular surface of the cell; and
(iv) a detectable moiety, a luminescent moiety, a Green Fluorescent Protein (GFP) or a luciferase, or any compound that can be directly or indirectly detected,
wherein the amino acid motif or subsequence susceptible to cleavage by the ZIKV protease or equivalent is positioned within the scaffold protein such that when the detectable moiety is cleaved away from (off from) the scaffold protein by the ZIKV protease or equivalent the remaining subsequence of scaffold protein on the extracellular surface of the cell lacks the detectable moiety;

(b) providing a nucleic acid encoding ZIKV protease or equivalent operatively linked to a transcriptional regulatory unit, or a cell that expresses a heterologous or endogenous ZIKV protease or equivalent;

(c) inserting (transfecting) the nucleic acid of (a) and (b) into the cell if the cell does not already express a heterologous or endogenous ZIKV protease or equivalent;

(d) co-expressing the nucleic acid of (a) and (b) in the cell, or expressing the nucleic acid of (a) in the cell if the cell already expresses a heterologous or endogenous ZIKV protease or equivalent; and

(e) determining whether the scaffold protein comprising the detectable moiety is expressed on the extracellular surface of the cell, optionally determined by a plate reader, a flow cytometry or any high-throughput assay,

wherein an intact scaffold protein comprising the detectable moiety is expressed on the extracellular surface of the cell when the ZIKV protease or equivalent is not enzymatically active, and an intact scaffold protein is not or is substantially less expressed on the extracellular surface of the cell when the ZIKV protease or equivalent is enzymatically active (the detectable moiety is cleaved off by the protease); or

(2) the cell-based platform, multiplexed platform or system, or cell-based method of (1), wherein the scaffold protein further comprises an endoplasmic reticulum (ER) retention motif or a KDEL motif,

wherein the ER retention motif or KDEL motif is positioned in the scaffold protein such that when the ZIKV protease or equivalent is active the scaffold will be separated into two pieces, leaving the ER retention motif-comprising or KDEL motif-comprising portion of the polypeptide in the ER and freeing the detectable moiety-comprising portion to the cell's extracellular membrane, and if the ZIKV protease or equivalent is blocked or inactive, the entire scaffold polypeptide will be retained in the ER, and as a consequence will not be detected on the cell's extracellular surface;

(3) the cell-based platform, multiplexed platform or system, or cell-based method of (1) or (2), wherein two or more, or a plurality of, ZIKV proteases or equivalents are screened in the same cell, wherein optionally they are variants of the same ZIKV protease or equivalent or a combination thereof;

(4) the cell-based platform, multiplexed platform or system, or cell-based method of any of (1) to (3), further comprising screening for an inhibitor of a ZIKV protease or equivalent by:
(a) providing a compound to be screened as an inhibitor of the ZIKV protease or equivalent, or providing a nucleic acid to be screened as encoding an inhibitor of the ZIKV protease or equivalent;
(b) contacting a plurality of the cells with the compound or nucleic acid either before, during and/or after the co-expressing the nucleic acid in the cell; and
(c) determining whether the scaffold protein comprising the detectable moiety is expressed on the extracellular surface of the cell,
wherein an intact scaffold protein comprising the detectable moiety is expressed on the extracellular surface of the cell when the ZIKV protease or equivalent is inhibited by: the compound, a composition encoded by the nucleic acid, or a compound present in the cell only because the nucleic acid was expressed, and an intact scaffold protein is not or is substantially less expressed on the extracellular surface of the cell when the ZIKV protease or equivalent is enzymatically active (the detectable moiety is cleaved off by the ZIKV protease or equivalent) and the enzymatic activity of the ZIKV protease or equivalent is not significantly inhibited by: the compound, a composition encoded by the nucleic acid, or a compound present in the cell only because the nucleic acid was expressed;

(5) the cell-based platform, multiplexed platform or system, or cell-based method of any of (1) to (4), further comprising:
(a) running a negative control comprising dividing the plurality of the cells co-expressing the nucleic acid of (a) and (b) in the cell and not adding the compound to be screened as an inhibitor to one of the divided cell samples; or
(b) running a positive control comprising dividing the plurality of the cells co-expressing the nucleic acid of (a) and (b) in the cell and adding a known inhibitor of the ZIKV protease or equivalent to one of the divided cell samples; or

(6) the cell-based platform, multiplexed platform or system, or cell-based method of any of (1) to (5), wherein
(a) the transcriptional regulatory unit comprises a promoter, or the transcriptional regulatory unit comprises an inducible promoter, or the transcriptional regulatory unit comprises a constitutive promoter;
(b) the cell is a mammalian cell, a monkey cell or a human cell; or a lymphocyte, or a T cell, or a CD4- or CD8-expressing cell, or a hepatocyte, and optionally the cells are genetically bar-coded;
(c) the scaffold proteins comprise all or part of a mouse Lyt2 or a human CD8 polypeptide;
(d) the detectable moiety comprises an epitope for an antibody, or a FLAG tag;
(e) the detectable moiety is detected or measured on the extracellular surface of the cell by a high throughput screen, a plate reader, a flow cytometry or a microscope visualization;
(f) the compound to be screened as an inhibitor of ZIKV protease or equivalent comprises a small molecule, a nucleic acid, a polypeptide or peptide, a peptidomimetic, a polysaccharide or a lipid;
(g) the compound to be screened as an inhibitor of ZIKV protease or equivalent is a member of a library of compounds to be screened, or a member of a random peptide library or a chemical compound library; or
(h) the ZIKV protease or equivalent further comprises its NS2B cofactor.

In alternative embodiments, provided are cell-based methods for monitoring the activity of a ZIKV protease or equivalent, comprising:

(a) providing a first chimeric nucleic acid encoding a chimeric protein, wherein the nucleic acid is operatively linked to a constitutive or an inducible transcriptional activator (optionally a constitutive or an inducible promoter),

where the chimeric protein comprises a proteolytically active ZIKV protease or equivalent and its cofactor polypeptide NS2B, wherein the NS2B polypeptide lacks its transmembrane domain (TM), and the proteolytically active ZIKV protease or equivalent is capable of recognizing and cleaving a specific cleavage site (CS), and the CS is positioned between the NS2B polypeptide and the proteolytically active ZIKV protease or equivalent,

and the proteolytically active ZIKV protease or equivalent and its cofactor polypeptide NS2B is positioned within the chimeric protein between two domains of a transcription factor comprising a DNA-binding domain (DBD) and a C-terminal Transactivation domain (TAD), wherein optionally the DBD and the TAD are derived from a yeast Gal4 protein transcription factor, and the transcriptional factor is active only if the DBD and the TAD are on or contained within the same chimeric protein,

and optionally the first nucleic acid comprises a construct as illustrated in FIG. 3 or FIG. 6, optionally a construct comprising: pH-TRE-Gal4-NS2B/NS3 Protease (pro) WT (wild type); or, pH-TRE-Gal4-NS2B/NS3 Pro WT 2CS (cleavage site),

and optionally the ZIKV NS3 protease has an amino acid sequence: ZIKV-2013 (SEQ ID NO:6); ZIKV-2007 (SEQ ID NO:7); ZIKV-1947 (SEQ ID NO:8);

and optionally the ZIKV NS3 Virus Polyprotein has a sequence comprising: NIBR Accession Number: AAV34151.1; NIBR Accession Number: ACD75819.1; or NIBR Accession Number: AHZ13508.1,

and optionally the ZIKV NS2B co-factors have amino acid sequences: ZIKV-2013 (SEQ ID NO:2); ZIKV-2007 (SEQ ID NO:3); ZIKV-1947 (SEQ ID NO:4);

and optionally the cleavage sites (CS) between NS2B/NS3, and NS3/NS4A for: ZIKV-2013=NS2B/NS3 (SEQ ID NO:9) and NS3/NS4A (SEQ ID NO:10); ZIKV-2007=NS2B/NS3 (SEQ ID NO:11) and NS3/NS4A (SEQ ID NO:12); ZIKV-1947=NS2B/NS3 (SEQ ID NO:13) and NS3/NS4A (SEQ ID NO:14); and/or (Dengue virus) DENV-2=NS2B/NS3 (SEQ ID NO:15) and NS3/NS4A (SEQ ID NO:16), where the “/” indicates the site of cleavage,

and optionally the cleavage site (CS) is a Zika Virus Polyprotein Cleavage Sites comprise:

Capsid C-Terminus Hydrophobic//Pre-membrane

| | |
|---|---|
| | (SEQ ID NO: 17) |
| MR-766 | 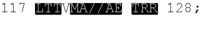 |
| | (SEQ ID NO: 18) |
| Yap-2007 | 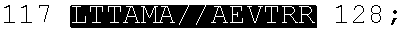 |
| | or |
| | (SEQ ID NO: 19) |
| FP-2013 | 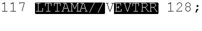 |

Pre-membrane//Membrane

| | |
|---|---|
| | (SEQ ID NO: 20) |
| MR-766 | 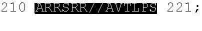 |
| | (SEQ ID NO: 21) |
| Yap-2007 | 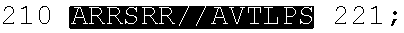 |
| | or |
| | (SEQ ID NO: 22) |
| FP-2013 | 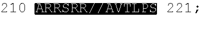 |

Membrane//Envelope

| | |
|---|---|
| | (SEQ ID NO: 23) |
| MR-766 | 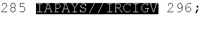 |
| | (SEQ ID NO: 24) |
| Yap-2007 | 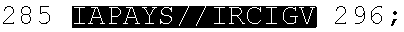 |
| | or |
| | (SEQ ID NO: 25) |
| FP-2013 | 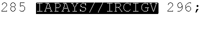 |

Envelope/NS1

| | |
|---|---|
| | (SEQ ID NO: 26) |
| MR-766 | 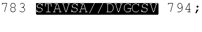 |
| | (SEQ ID NO: 27) |
| Yap-2007 | 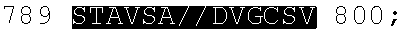 |
| | or |
| | (SEQ ID NO: 28) |
| FP-2013 | 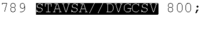 |

NS1//NS2A

| | |
|---|---|
| | (SEQ ID NO: 29) |
| MR-766 | 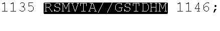 |
| | (SEQ ID NO: 30) |
| Yap-2007 | 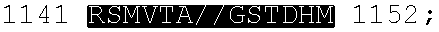 |
| | or |
| | (SEQ ID NO: 31) |
| FP-2013 | 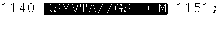 |

NS2A//NS2B

| | |
|---|---|
| | (SEQ ID NO: 32) |
| MR-766 | 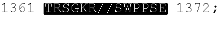 |
| | (SEQ ID NO: 33) |
| Yap-2007 | 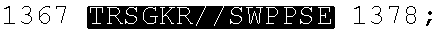 |
| | or |
| | (SEQ ID NO: 34) |
| FP-2013 | 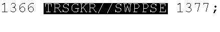 |

NS2B//NS3

| | |
|---|---|
| | (SEQ ID NO: 35) |
| MR-766 | 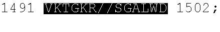 |
| | (SEQ ID NO: 36) |
| Yap-2007 | 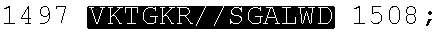 |
| | or |
| | (SEQ ID NO: 37) |
| FP-2013 | 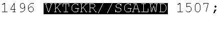 |

NS3//NS4A

| | |
|---|---|
| | (SEQ ID NO: 38) |
| MR-766 | 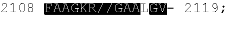 |
| | (SEQ ID NO: 39) |
| Yap-2007 | 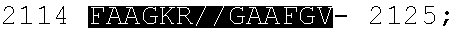 |
| | or |
| | (SEQ ID NO: 40) |
| FP-2013 | 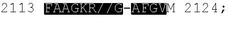 |

NS4A//2K

| | |
|---|---|
| | (SEQ ID NO: 41) |
| MR-766 | 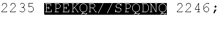 |
| | (SEQ ID NO: 42) |
| Yap-2007 | 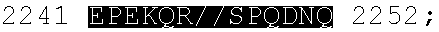 |
| | or |
| | (SEQ ID NO: 43) |
| FP-2013 | 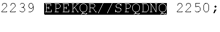 |

2K//NS4B

| | |
|---|---|
| | (SEQ ID NO: 44) |
| MR-766 | 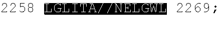 |
| | (SEQ ID NO: 45) |
| Yap-2007 | 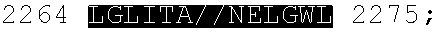 |
| | or |
| | (SEQ ID NO: 46) |
| FP-2013 | 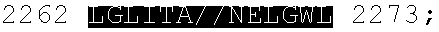 |
| | or |

NS4B//NS5

| | |
|---|---|
| | (SEQ ID NO: 47) |
| MR-766 | 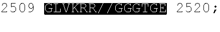 |
| | (SEQ ID NO: 48) |
| Yap-2007 | 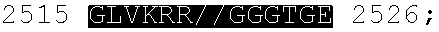 |
| | or |
| | (SEQ ID NO: 49) |
| FP-2013 | 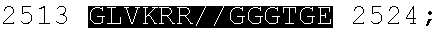 |

(b) providing a second chimeric nucleic acid encoding a detectable moiety or a detectable protein, wherein the nucleic acid is operatively linked to a promoter activated by the transcription factor encoded by the first chimeric nucleic acid of (a),

and optionally the detectable moiety or the detectable protein is or comprises a fluorescent protein or a luminescent protein, and optionally the fluorescent protein is a Green Fluorescent Protein (GFP), an enhanced Green Fluorescent Protein (eGFP), or a luciferase,

and optionally the second chimeric nucleic acid is contained in a same or a different construct as the first chimeric nucleic acid, wherein optionally the construct is a plasmid, a viral vector or a retroviral vector,

(c) inserting or transfecting the first chimeric nucleic acid of (a) and the second chimeric nucleic acid of (b) into a cell, wherein if the first chimeric nucleic acid of (a) and the second chimeric nucleic acid of (b) are on the same construct, then inserting or transfecting the construct into the cell,

wherein optionally the cell is genetically bar-coded;

(d) co-expressing the first chimeric nucleic acid of (a) and the second chimeric nucleic acid of (b) in the cell; and

(e) determining whether the detectable moiety or the detectable protein is expressed in the cell,

wherein if the ZIKV protease or equivalent is active it will cleave the chimeric protein at the cleavage site or cleavage sites and separate the DBD and the TAD, and as a result the transcription factor, lacking a DBD and an TAD on the same chimeric protein, cannot bind to and activate the promoter on the second chimeric nucleic acid to result in transcription of the detectable moiety or the detectable protein, and thus the cells will not express the detectable moiety or the detectable protein, and if the detectable moiety or the detectable protein is a fluorescent or a luminescent protein, the cell will not fluoresce or luminesce,

and if the ZIKV protease or equivalent is inactive or inactivated, the cells will express the detectable moiety or the detectable protein, and if the detectable moiety or the detectable protein is a fluorescent or a luminescent protein, the cell will fluoresce or luminesce.

In alternative embodiments, provided are cell-based methods for screening of an activator or inhibitor of a ZIKV protease or equivalent, comprising: a method as provided herein further comprising exposing the cell, or adding to the cell, or expressing in the cell, a potential, putative or candidate inhibitor or activator of the ZIKV protease or equivalent,

wherein if the ZIKV protease or equivalent is inactivated by the potential, putative or candidate inhibitor, the cells will express the detectable moiety or the detectable protein, and if the detectable moiety or the detectable protein is a fluorescent or a luminescent protein, the cell will fluoresce or luminesce.

In alternative embodiments, the potential, putative or candidate inhibitor or activator of the ZIKV protease or equivalent to be screened comprises: a small molecule, a nucleic acid, a polypeptide or peptide, a peptidomimetic, a polysaccharide or a lipid; is a member of a library of compounds to be screened, or is a member of a random peptide library or a chemical compound library.

In alternative embodiments, methods as provided herein further comprise:

(a) running a negative control comprising dividing a plurality of the cells co-expressing the first chimeric nucleic acid of (a) and the second chimeric nucleic acid of (b) in the cell and not adding the potential, putative or candidate inhibitor or activator of the ZIKV protease or equivalent to be screened to one of the divided cell samples; or

(b) running a positive control comprising dividing the plurality of the cells co-expressing the first chimeric nucleic acid of (a) and the second chimeric nucleic acid of (b) in the cell and adding a known inhibitor of the ZIKV protease or equivalent to one of the divided cell samples.

In alternative embodiments, provided herein are recombinant or engineered isolated cells comprising the first chimeric nucleic acid or the first chimeric nucleic acid and the second chimeric nucleic acid used in a method as provided herein.

In alternative embodiments, provided herein are constructs comprising or having contained therein a nucleic acid encoding the first chimeric nucleic acid or the first chimeric nucleic acid and the second chimeric nucleic acid used in a method as provided herein, wherein optionally the construct is a plasmid, a vector or a recombinant vector.

The details of one or more embodiments of the invention are set forth in the accompanying drawings and the description below. Other features, objects, and advantages embodiments provided herein will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1-B graphically illustrate a representation of exemplary assays provided herein. FIG. 1A illustrates Wild type Gal4 as control, without Dox. In the absence of Dox, rtTA cannot bind to the Tet-Responsive Element (TRE) and consequently, there is no GFP expression from the reporter construct. In the presence of Dox, rtTA binds to TRE and induces GFP expression. FIG. 1B illustrates the protease (PR)/Gal4 fusion-based system. In the presence of Dox, PR/Gal4 is expressed; however, its autocatalytic activity results in the separation of the Gal4 domains, resulting in turn in the lack of GFP expression. However, in the presence of protease inhibitor (PI), the PR/Gal4 fusion remains intact, resulting in the induction of GFP expression. The same result is expected with an inactive mutant PR in the absence of inhibitor, as further discussed, below, see e.g., Example 1.

FIGS. 2A and 2B illustrate the establishment of cell lines and demonstration of assay capabilities; FIG. 2A graphically illustrates an exemplary vector used to corroborate the expression of a scaffold provided herein on a cell surface by flow cytometry, as graphically illustrated in FIG. 2B. FIG. 2A illustrates retroviral plasmids utilized for the production of mammalian cell lines carrying the various elements of the assay. The plasmids include the rtTA, GFP reporter and Gal4 fusions. The Gal4 plasmids include Gal4 control, Gal4/PRm (mutant) and Gal4/PR. FIG. 2B illustrates flow cytometry-based quantification of GFP expression of each cell line, demonstrating the ability to monitor cleavage in an inducible manner. The no-treatment column shows that the cell populations are non-fluorescent in the absence of Dox. However, when dox is added, a drastic shift occurs with the positive Gal4 control in the absence or presence of the PI Indinavir (IDV). Importantly, while PRm behaves similarly to the Gal4 control, the PR cell line is not green when induced by Dox, unless it is also incubated with the PI IDV, as further discussed, below, see e.g., Example 1.

FIG. 3A-B illustrate structures of exemplary ZIKV-based constructs in the context of Gal4. Plasmid constructs that are used for the ZIKV assay include NS2B/NS3 fusions with full, Δ2TM or Δ4TM NS2B cofactor with full or protease domain-only NS3, as further discussed, below, see e.g., Example 1.

FIG. 4A-B illustrate amino acid sequence homology between NS2B co-factor and NS3 proteases of different ZIKV strains and Dengue virus, as further discussed, below; proteases are:

FIG. 4A illustrates, the ZIKV protease NS2B co-factor amino acid sequences: ZIKV-2013 (French Polynesia Strain) (SEQ ID NO:2); ZIKV-2007 (Micronesia Strain) (SEQ ID NO:3); ZIKV-1947 (YAP Ethiopia strain) (SEQ ID NO:4); and (Dengue virus) DENV-2 (SEQ ID NO:5),

FIG. 4B illustrates, the ZIKV N3 protease: ZIKV-2013 (French Polynesia Strain) (SEQ ID NO:6); ZIKV-2007 (Micronesia Strain) (SEQ ID NO:7); ZIKV-1947 (YAP Ethiopia strain) (SEQ ID NO:8).

FIG. 5 illustrates in a table format the amino acid sequences of the cleavage sites (CS) between NS2B/NS3, and NS3/NS4A for: ZIKV-2013=NS2B/NS3 (SEQ ID NO:9) and NS3/NS4A (SEQ ID NO:10); ZIKV-2007=NS2B/NS3 (SEQ ID NO:11) and NS3/NS4A (SEQ ID NO:12); ZIKV-1947=NS2B/NS3 (SEQ ID NO:13) and NS3/NS4A (SEQ ID NO:14); and (Dengue virus) DENV-2=NS2B/NS3 (SEQ ID NO:15) and NS3/NS4A (SEQ ID NO:16), where the “I” indicates the site of cleavage.

FIG. 6 illustrates the structure of exemplary ZIKV-based constructs, as discussed in detail in Example 2, below.

FIG. 7 illustrates an image of a Western blot that demonstrates that exemplary constructs of FIG. 6 carry the protease fusion as described by demonstrating their expression in cells; experiments detected the fusion protein in cells transduced/infected (lane 2 and 3) or transfected (lane 4 and 5), as discussed in detail in Example 2, below.

FIG. 8A-B and FIG. 9 illustrate an image of a flow cytometry study where cells were transfected with the different exemplary constructs of FIG. 6 in order to corroborate their effect on GFP activation: cells were analyzed 48 hours post-transfection; results of control are in FIG. 8A and results from using experimental constructs are in FIG. 8B and FIG. 9, as discussed in detail in Example 2, below.

FIG. 10 illustrates the domains and in vivo plasma membrane configuration of a ZIKA virus.

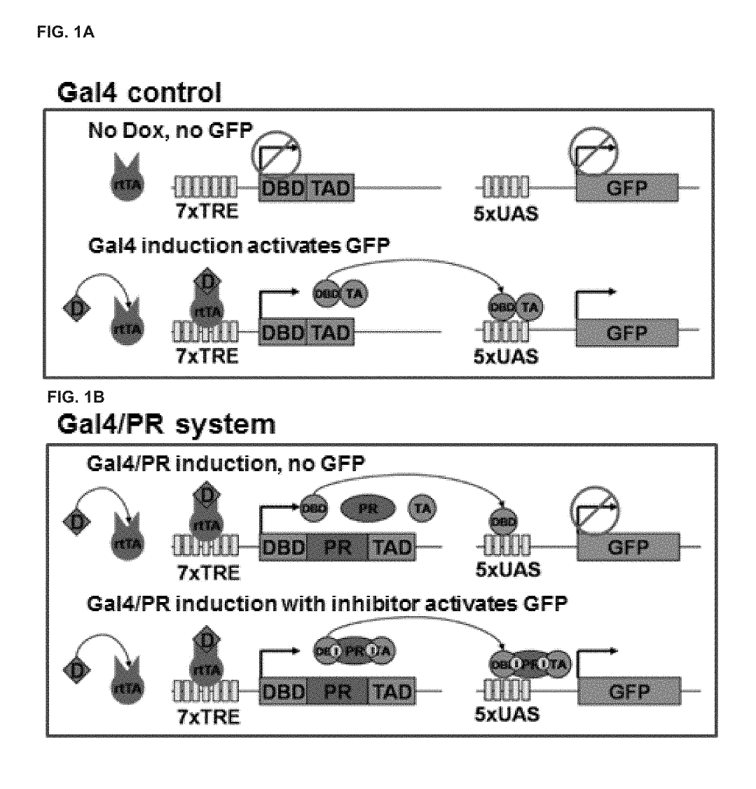

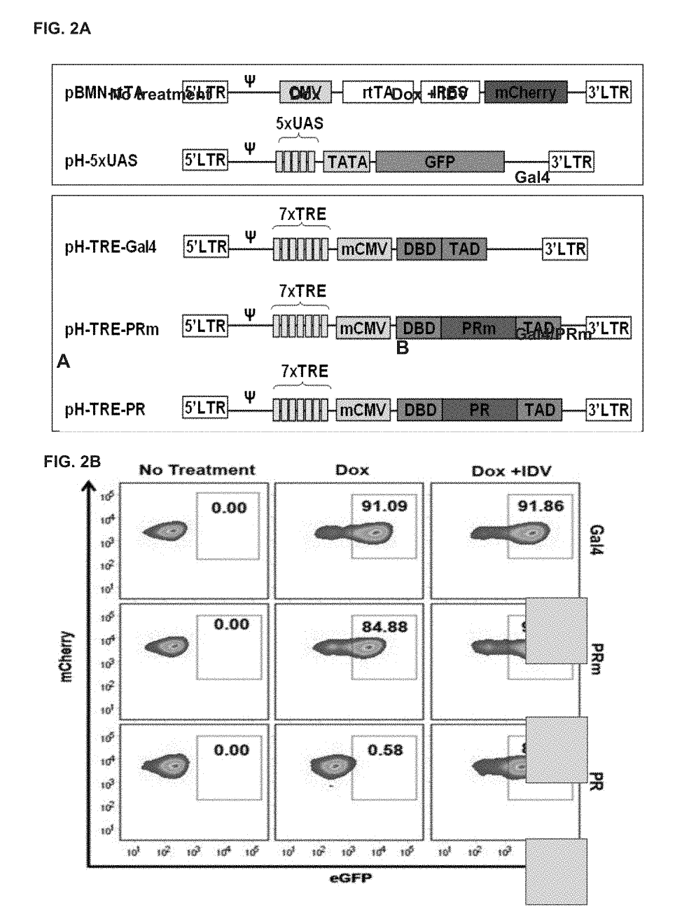

Like reference symbols in the various drawings indicate like elements.

### DETAILED DESCRIPTION

In alternative embodiment, provided herein are methods and compositions, including chimeric recombinant proteins, nucleic acids that encode them, and cells and kits comprising them, to screen for compositions, e.g., small molecule drugs, that can modulate, e.g., inhibit or enhance, Zika Virus (ZIKV) proteases.

In an effort to most affectively mimic the biological context of the engineered a Zika Virus (ZIKV) protease, e.g., a ZIKV NS3 protease, natural topology into the ER multiple constructs are provided, each utilizing specific aspects of the protein such as natural hydrophobic regions within the protein that will localize and anchor it within the ER. Mutated NS3 catalytic-domain control constructs are also engineered by site directed mutations. The cell-based assay is usable for drug discovery as it monitors cleavage in biologically relevant cells, and can also determine whether or not a specific inhibitor targets the viral protease while not affecting the natural processes of the host, which would lead to cytotoxic side effects.

We have developed a unique cell-based platform that monitors proteolytic cleavage, including Zika Virus (ZIKV) protease cleavage, that is robust and generally applicable to antiviral drug discovery. We initially developed and validated the technology as a platform that facilitates HIV-1 protease and HIV-1 envelope targeted drug discovery, and have now adapted the platform to target Zika Virus (ZIKV) protease. The platform leverages and combines the power of retroviral technology for stable expression of viral targets in mammalian cells and the utility of flow cytometry to detect fluorescent proteins. We have used Dengue targeted assays to conduct a pilot screen and have a potential screening hit in hand. Easily adaptable to other important human viral pathogens including Chikungunya and Zika, or their associated serotypes, our cell-based platforms are engineered to target particular cellular compartments within mammalian cells. The ability to multiplex targets (e.g.: viral protease/viral proteome cleavage by the host) raises the value of the platform for screening by increasing selectivity in the right cellular context, thus improving hit-to-lead compound discovery. In addition, the appropriate cellular/subcellular nature of our adaptable platform also reveals information about drug cytotoxicity and permeability.

Systems and methods provided herein are mammalian in nature, so in alternative embodiments, proteins are expressed in cells through retroviral technology. This can be very important, as the expression of constructions and proteins in mammalian cells allows for the possible post-translational modifications of viral proteins that might be required for full activity, as well as giving the cellular environment and cellular factors required. This is in contrast with bacterial/recombinant in vitro assays.

In alternative embodiments, systems and methods provided herein are adapted for the expression of the NS2B/NS3 ZIKV protease sequence (co-factor NS2B and protease NS3) with three different flavors:
1—In fusion with the Gal4 system for continuous expression;
2—In fusion with the Gal4 system in an inducible expression; and
3—By themselves, not in fusion with Gal4, for testing their effects on cells, serving also as control in exemplary systems.

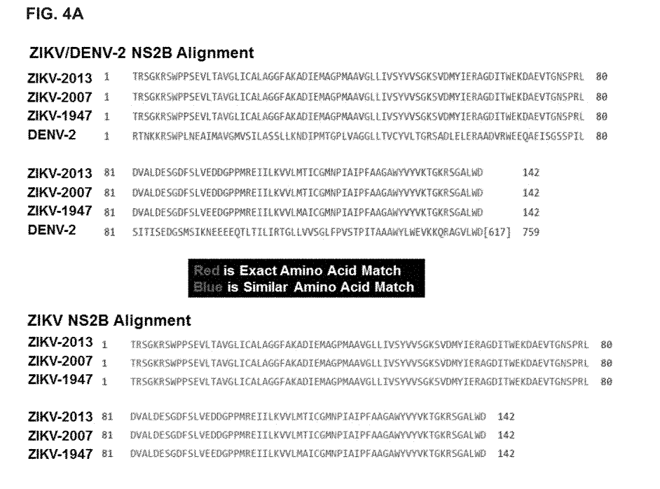

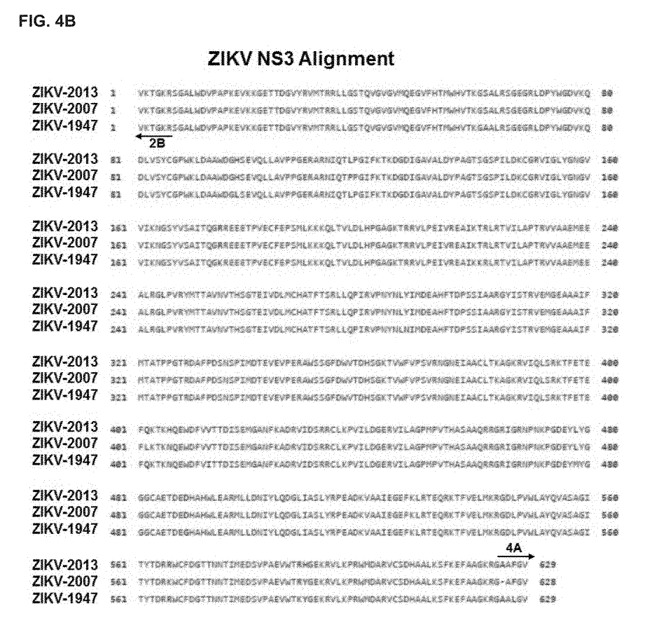

The prototype of the ZIKV protease engineered for assays provided herein is based on the sequence of the 2015 Brazilian strain, which is 100% homologue to the 2013 French Polynesia strain shown in FIG. 4, where only one amino-acid change has been observed in another protein, NS5, and none in NS2B/NS3. Importantly, as shown in FIG. 4, the sequence homology between ZIKV and Dengue is high (with around 55% exact amino acids and 72% similar amino acids (top of FIG. 4). The similarity among ZIKV strains is more than 99% so older strains (1947 Uganda and 2007 Micronesia) have minimal changes when compared to French Polynesia (and thus Brazil). The sequence comparison of the NS3 protease among the strains is shown on the bottom of FIG. 4.

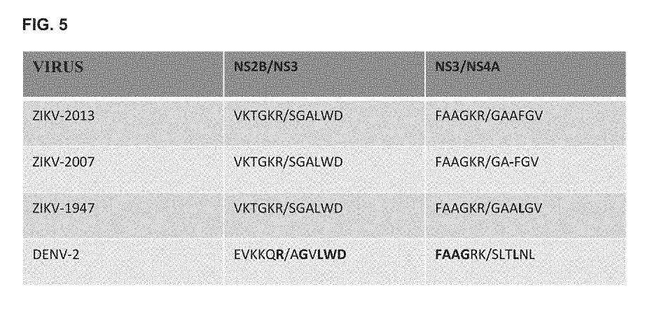

FIG. 4 also illustrates the boundaries between NS2B/NS2A, and NS3/NS4A. The cleavage sites between NS2B/NS3, and NS3/NS4A, which are included in our assay for the 2013 French Polynesia strain (or Brazilian strain), are shown in FIG. 5.

In alternative embodiments, if/when mutations at an RNA sequence and/or amino acid sequence are observed in ZIKV, e.g., an important ZIKV strain or serotype, those can be easily incorporated into the assays provided herein in such a way that the assay best monitors the activity of the viral protease and co-factor, as well as providing the best fit cellular platform for drug discovery.

In alternative embodiment, assays provided herein can discriminate between fluorescent and non-fluorescent cells. These platforms can monitor cleavage in a robust and reliable manner. The assays rely on the appearance of fluorescence when protease activity is inhibited, which is a valuable asset for straightforward screening efforts. Fluorescence can be detected by flow cytometry or fluorescent microscopy, and can be easily adapted to non-fluorescent luminescence for other plate-reader setups. In alternative embodiment, assays provided herein are miniaturized in 96 well-plate formats, and can be adapted to 384 and 1536 well-plate formats to drastically enhance drug screening in an unexploited market.

In one embodiment, these cells and cell-based assays are used to screen for and identify novel Zika Virus (ZIKV) protease inhibitors. In one embodiment, cell-based platforms and assays provided herein effectively couple the surface (extracellular) expression of a protein used as a scaffold (a scaffold protein), with the activity of the protease (e.g. viral protease).

In one embodiment, the scaffold is engineered for its conditional expression on the surface of a cell, e.g., a eukaryotic, a yeast or a mammalian cell. In alternative embodiments, the cell or cells are or comprise lymphocytes, e.g., T cells, or hepatocytes or equivalent cells. For that purpose, in one embodiment, the scaffold is fused to a signal sequence to enable efficient and/or directed transport, and a transmembrane domain (e.g., an Lyt2, the murine CD8 molecule, and the like) is used to enable subsequent insertion in the cell membrane. In one embodiment, a tag such as a FLAG tag is added to the scaffold downstream of the signal sequence for detection, e.g., for antibody detection, e.g., through flow cytometry or equivalent visualization.

In one embodiment, the assay co-expresses both the scaffold protein and the ZIKV protease or equivalent, which if active will bind to and cleave the scaffold at the protease recognition sequence.

In alternative embodiments, both scaffold and ZIKV protease or equivalent are co-expressed in a cell, e.g., a lymphocyte such as a T cells, e.g., SupT1 T-cells, or a hepatocyte, in an inducible off/on-based vector system (e.g., activated upon addition of tetracycline or doxycycline). Inducible expression of protease can help avoid its possible cytopathic effects. Inducible expression of the scaffold may be necessary as a protease will only be able to prevent surface expression of newly synthesized intact scaffold, as a pre-inserted scaffold would not be removed from the cell surface by the protease.

In one embodiment, the logic behind the engineering of the scaffold as a membrane-expressed protein is as follows: in the presence of the active protease, e.g. viral protease, the proteolytic enzyme will cleave the scaffold, resulting in the loss of transmembrane domain, thus preventing tag cell surface expression. In the absence of protease, or when protease is blocked or inhibited, the scaffold will be intact and incorporated into the membrane. As a result, the surface expression of the scaffold can be determined by flow cytometry allowing the discrimination between active and inactive or blocked protease. In one embodiment, the platform or assay is cell-based, and can be easily implemented for a high throughput screen, e.g., FACS. As such, this platform or assay is invaluable for drug discovery, and can be utilized in biological screens aimed at finding novel enzyme or protease inhibitors through random peptide libraries or chemical compounds libraries.

In one embodiment, provided are engineered protein scaffolds bearing the protease cleavage site on the cell surface of a mammalian cell (e.g., a lymphocyte such as a T cell, or a hepatocyte). In one embodiment, ZIKV protease or equivalent is expressed, or co-expressed on a scaffold used as a target, in an inducible manner (the protease, the scaffold, or both can be expressed via an inducible mechanism, e.g., an inducible transcriptional regulator).

In one embodiment, provided herein are assays that can be adapted for a high throughput manner using e.g. flow cytometry such as FACS, and can discriminate between active and non-active or blocked protease. In one embodiment, provided herein are assays that can be easily adapted for high throughput screening. In one embodiment, provided herein are assays that can be used to screen for novel ZIKV protease or equivalent inhibitors.

In one embodiment, provided herein are assays provided herein adapted for the screen of random peptide libraries or chemical compounds for drug discovery.

In one embodiment, the methods provided herein use a random peptide library or any peptide of choice, which can be introduced ‘in cis’, replacing the a known Zika Virus (ZIKV) protease recognition/cleavage site, enabling the discovery of higher affinity sites for PR, which can be the basis for the development of competitor peptidomimetic drugs. In one embodiment, the random peptide library is expressed ‘in trans’, enabling the discovery of competitors/inhibitors for ZIKV protease or equivalent (PR), which can be the basis for peptidomimetic drugs.

In one embodiment, the non-biased approach provided herein permits the rescue of peptides or chemicals targeted not necessarily to the catalytic site of PR. Thus, the assays provided herein provide for extensive characterization of PR, facilitating the elucidation of interactions of PR with cellular targets, its mode of action and modulation, in the context of the host cell. Assays provided herein will permit the replacement of PR with PR from different viral strains or clades, or truncated versions of PR, enabling further dissection of PR activity, and study its modulation through co-expression of cellular factors or addition of drugs.

In alternative embodiments, the assays provided herein comprise expression of a scaffold naturally expressed in the cytoplasm that is able to be exported into the cell membrane.

In alternative embodiments, assays provided herein comprise expression of both PR and scaffold in an off/on system for inducible expression.

In alternative embodiments, assays provided herein comprise expression of a protein that is expressed on the surface of the mammalian cell (e.g., a lymphocyte such as a T cell, or a hepatocyte) only when not cleaved by a Zika Virus (ZIKV) protease.

In alternative embodiments, assays provided herein can be adapted for the screen of random peptide libraries or chemical compounds.

In alternative embodiments, assays provided herein can be implemented in mammalian cells (e.g., a lymphocyte such as a T cell, or a hepatocyte) and other cells, e.g., yeast or bacterial cells.

The Expression of Protease in a Non-Toxic Inducible Manner.

In one embodiment, to achieve low levels of ZIKV protease or equivalent expression in mammalian cells (e.g., a lymphocyte such as a T cell, or a hepatocyte), a tetracycline (Tet) inducible system is used. Enhanced green fluorescent protein (eGFP) can be used as the ectopic gene. Use of this vector allows different levels of protein expression. Tight repression and expression of ZIKV protease or equivalent (PR) at low levels may be crucial to avoid the possible side effects of PR. It is important to mention that this system is an off/on system that allows for expression of the gene of interest only upon addition of tetracycline or doxycycline. The inducible system allows for de novo synthesis of the scaffold, needed for the successful implementation of the assay.

An exemplary scaffold provided herein is an adapted scaffold based on the same idea, but with an important difference. In one embodiment, when ZIKV protease or equivalent (PR) is active the FLAG or other detectable tag will be present on the surface and detectable e.g., by flow cytometry, whereas when protease (e.g., PR) is blocked or inactive, the FLAG will be lost and not expressed on the cell surface. This scaffold is based on the idea that generally, proteins to be expressed on the surface of the cell have a signal sequence (SS) on their N terminus that targets them to the endoplasmic reticulum (ER) and a transmembrane domain (TM) that retains them in the membrane.

On the other hand, proteins that are retained in the ER, will have, in addition to the SS and the TM, an ER-retention signal such as the prototypic KDEL sequence. This sequence is known to have strong affinity to the KDEL receptor (SEQ ID NO:1), acting the luminal side of the ER. In one embodiment, the ER retention motif or KDEL (SEQ ID NO:1) motif is positioned in the scaffold protein such that when PR is active the scaffold will be separated into two pieces, leaving the ER retention motif-comprising or KDEL (SEQ ID NO:1) motif-comprising portion of the polypeptide in the ER and freeing the detectable moiety-comprising portion to the cell's extracellular membrane, and if PR is blocked or inactive, the entire scaffold polypeptide will be retained in the ER, and as a consequence will not be detected on the cell's extracellular surface

For example, an exemplary CCR5 engineered protein (or partial CCR5) can be replaced by any other protein of choice or hybrid protein. For example, one exemplary embodiment comprises a hybrid protein comprising the N-terminus of the CD8 molecule or the CD8 molecule equivalent in mice (referred to as Lyt2), comprising its natural SS, and the C-terminus of the chemokine receptor CCR5 including only the last TM domain. In this embodiment, only requirement is that the resulting protein will, when cleaved, retain the KDEL-containing side in the ER and the N-terminus on the cell surface.

In alternative embodiments, any protein is used as scaffold (instead of the exemplary proteins described herein), provided that by adding a KDEL sequence at the C terminus the polypeptide will be retained it in the ER, unless separated from the N-terminus.

In alternative embodiments, the scaffold is engineered for the search of ZIKV protease or equivalent inhibitors (active in the cytoplasm). In one embodiment, the loop facing the luminal face of ER is substituted by a recognition site cleaved by cellular peptidases.

In alternative embodiments, provided are methods and compositions, including chimeric recombinant proteins, nucleic acids that encode them, and cells and kits comprising them, to screen for compositions, e.g., small molecule drugs, that can modulate, e.g., inhibit or enhance, ZIKV protease or equivalent.

In one embodiment, provided herein are cells and cell-based assays for monitoring the activity of activity ZIKV protease or equivalent. In one embodiment, these cells and cell-based assays are used to screen for and identify novel ZIKV protease or equivalent inhibitors. In one embodiment, assays provided herein effectively couple the surface (extracellular) expression of a protein used as a scaffold (a scaffold protein), with the activity of the ZIKV protease or equivalent (PR).

In one embodiment, the scaffold is engineered for its conditional expression on the surface of a cell, e.g., a yeast or a mammalian cell (e.g., a lymphocyte such as a T cell, or a hepatocyte). For that purpose, in one embodiment, the scaffold is fused to a signal sequence to enable efficient transport, and a transmembrane domain (e.g., an Lyt2, the murine CD8 molecule, and the like) is used to enable subsequent insertion in the cell membrane. A tag such as a FLAG tag is added to the scaffold downstream of the signal sequence for detection, e.g., for antibody detection, e.g., through plate-reader, flow cytometry or equivalent visualization, or any similar or equivalent detection system.

In one aspect, both scaffold and protease are co-expressed in a lymphocyte, e.g., a T cell or T cells, e.g., a SupT1 T-cell, in an inducible off/on-based vector system (e.g., activated upon addition of tetracycline or doxycycline). Inducible expression of PR helps avoid its possible cytopathic effects. Inducible expression of the scaffold is necessary as PR will only be able to prevent surface expression of newly synthesized intact scaffold, as the pre-inserted scaffold would not be removed from the cell surface by PR.

### Kits

Provided are kits comprising compositions and instructions for use in practicing compositions and methods as described herein. The kits can include: cells comprising nucleic acids encoding the chimeric polypeptides provided herein (the “scaffold proteins”) and/or vectors comprising these nucleic acids, or chimeric polypeptides as provided herein, transfecting agents, transducing agents, instructions (regarding the methods as provided herein), or any combination thereof. As such, kits, cells, and libraries of compounds are provided herein.

### Cell-Based Methods and Multiplexed Systems

In alternative embodiments, provided herein are cells and cell-based assays and multiplexed systems for monitoring the activity of activity of ZIKV protease or equivalent. In one embodiment, these cells and cell-based assays are used to screen for and identify novel ZIKV protease or equivalent (PR) inhibitors (“PIs”). In one embodiment, provided herein are methods and compositions, including chimeric recombinant proteins, nucleic acids that encode them, and cells and kits comprising them, to screen for compositions, e.g., small molecule drugs, that can modulate, e.g., inhibit or enhance, ZIKV proteases or equivalents.

In one embodiment, provided herein are assays and multiplexed systems in T cells to monitor the proteolytic activity of ZIKV protease or equivalent. The assay is based on an inducible Gal4-PR fusion which binds to upstream activation sequences and activates a reporter gene only in the presence of a PR inhibitor (“PI”).

In one embodiment, provided herein are clones which, when activated, express eGFP as a biosensor of PR activity. This assay has a robust and reliable readout that relies on green fluorescence, making it ideal for high-throughput screening utilizing flow cytometry. Thus, the assay provided herein will greatly facilitate the search for novel peptide- and chemical-compound-based PIs in T-cells.

In one embodiment, provided herein is a simple, rapid and straightforward method and multiplexed systems for monitoring a ZIKV protease or equivalent (PR) activity to facilitate the search for novel inhibitors/competitors of the protease that could lead to new therapeutics, e.g., to treat ZIKV.

In one embodiment, assays provided herein are based on the classical Gal4-UAS system, a broadly utilized system for the analysis of gene expression. The yeast Gal4 protein represents a prototypic transcription factor consisting of two separate domains: An N-terminal DNA-binding domain (DBD: aa 1-147) and a C-terminal Transactivation domain (TAD: aa 768-881). The Gal4 protein binds to consensus Upstream Activation Sequences (UAS's) via its DBD and activates transcription of downstream genes through its TAD. However, when the two Gal4 domains are separated, neither half of the protein can independently serve as a functional transcription factor.

Murray (1993) Gene 134(1):123-128, demonstrated the ability for ZIKV protease or equivalent fused within Gal4 to auto-catalytically remove itself, leaving behind the two non-functional domains of Gal4. When the PR/Gal4 fusion protein is mutated at the catalytic site, however, or is in the presence of an inhibitor, the fusion protein remains intact, retaining its ability to bind to UAS through the DBD and activate transcription through TAD. In alternative embodiments, this property is incorporated into embodiments provided herein to express a reporter gene in an inversely proportional manner to PR activity and serve as template for this assay.

In alternative embodiments, assays and multiplexed systems as provided herein are based on the expression of the ZIKV protease or equivalent (PR)/Gal4 fusion as an inducible fusion through a Tet-On system (e.g., in one embodiment, adapted from Clontech, Takara Bio Inc., Shiga, Japan), thus drastically reducing its possible toxic side effects. In this embodiment, the reverse tetracycline transactivator (rtTA) is utilized, allowing for the induction of PR/Gal4 expression only upon addition of tetracycline (Tet) or doxycycline (Dox). The readout; eGFP expression, will appear only when PR/Gal4 expression is induced in the presence of inhibitor. Moreover, all the elements of the assay have been constructed in retroviral vectors for their stable expression in mammalian cells. In alternative embodiments, the assays provided herein are designed for use in lymphocytes such as T cells, or hepatocytes, to facilitate the high-throughput screening for novel inhibitors in a more natural milieu.

In alternative embodiments, assays provided herein are adapted such that the cells carry several enzymes, including Zika Virus (ZIKV) protease and mutant variants, including for example a PR mutant shown to be resistant to a known inhibitor, e.g., an FDA-approved inhibitor. In alternative embodiments, clones comprising different enzymes (e.g., PRs), when inhibited, activate the transcription of a different fluorescent marker. Accordingly, in alternative embodiments, exemplary assays provided herein are adapted as multiplexed systems.

Clones provided herein are valuable for the screening of inhibitors against the specific PR used in the assay; from the HXB2 consensus T-tropic strain. Due to the high mutational rate it is crucial to adapt the assay to as many protease variants as possible—and the assays and multiplexed systems provided herein are adaptable to multiple protease variants. In alternative embodiments, assays provided herein are adapted to an array of proteases. In alternative embodiments, these assays are configured as multiplexed systems provided herein.

In alternative embodiments, assays provided herein are adapted to multiplexed formats with ZIKV protease or equivalent (PR) mutants/variants and reporter combinations to simultaneously detect ZIKV protease or equivalent resistance to individual hits. In alternative embodiments, assays provided herein are adapted to Luminescence/plate reader-based formats. In alternative embodiments, assays provided herein are adapted to screening peptide and chemical-compound libraries.

The invention will be further described with reference to the following examples; however, it is to be understood that embodiments provided herein not limited to such examples.

### EXAMPLES

### Example 1: Exemplary Assays and Constructs

In alternative embodiments, provided are compositions (including exemplary constructs) and assays for screening for Zika Virus (ZIKV) protease, e.g., a ZIKV NS3 protease, inhibitors.

Provided herein are assays to monitor the activity of the Zika Virus protease in cells. Provided herein are assays to monitor the cleavage of the boundaries between NS2B/NS3 and NS3/NS4A of ZIKV. The cleavage recognition sites within the boundaries are composed of six specific amino acids at both C- and N-terminal sides of the NS3 protease. Two main outcomes are possible: either the expression of the GFP reporter gene is expressed in the absence of cleavage, or conversely, it is not expressed in the presence of cleavage.

In order to be able to easily discriminate between cleaved and non-cleaved events (or active and non-active protease), the assay relied on the classical Gal4/UAS system broadly used in gene expression experiments. Gal4 is a prototypic yeast transcription factor that contains two well-defined domains: a 5′DNA-binding domain (DBD) and a 3′ Transactivating domain (TAD). The assay is based on the expression of a Gal4/protease (PR) fusion where PR is inserted between the two Gal4 domains. The fusion protein is expressed in an inducible manner via the Tet-On system, in order to control its expression and the possible cytotoxic effects of when over-expressed. Inducibility is achieved with the reverse tetracycline transactivator (rtTA) with the addition of tetracycline or doxycycline (Dox). When the antibiotic binds to rtTA it causes a conformational change in rtTA that enables it to bind to the tetracycline response element (TRE) on the Gal4/PR plasmid, thus driving expression of the Gal4/PR fusion. In the absence of cleavage the intact Gal4/PR fusion retains the DNA-binding properties of DBD and the transactivation properties of TAD, allowing the fusion to travel to the nucleus, bind to the upstream activation sequence (UAS), and drive the expression of the GFP reporter.

However, when PR autocatalytically cleaves itself out of the Gal4/PR fusion, DBD and TAD are separated. In this scenario, while DBD travels to the nucleus and binds UAS, the reporter is not transactivated. Naturally, no expression of GFP then occurs. A Gal4-only construct, consisting of DBD and TAD with no PR sequence, is used as control. Gal4 serves as control for both inducibility of the system and lack of cleavage (as no protease is present). With the Gal4 control, cells are not green in the absence of Dox as no protein is expressed to transactivate the GFP reporter gene. However, in the presence of Dox, Gal4 is expressed and as expected, a strong induction of GFP is observed. The expression or lack thereof of the reporter gene GFP is quantified via flow cytometry.

This exemplary system is robust and clearly demonstrates the ability to monitor PR activity. We have demonstrated that while the Gal4/PR does not activate GFP expression when induced, it does so when a PI is added. Similar results were obtained with all FDA-approved inhibitors, demonstrating the usability of the cellular platform to monitor PR activity.

Experimental Design

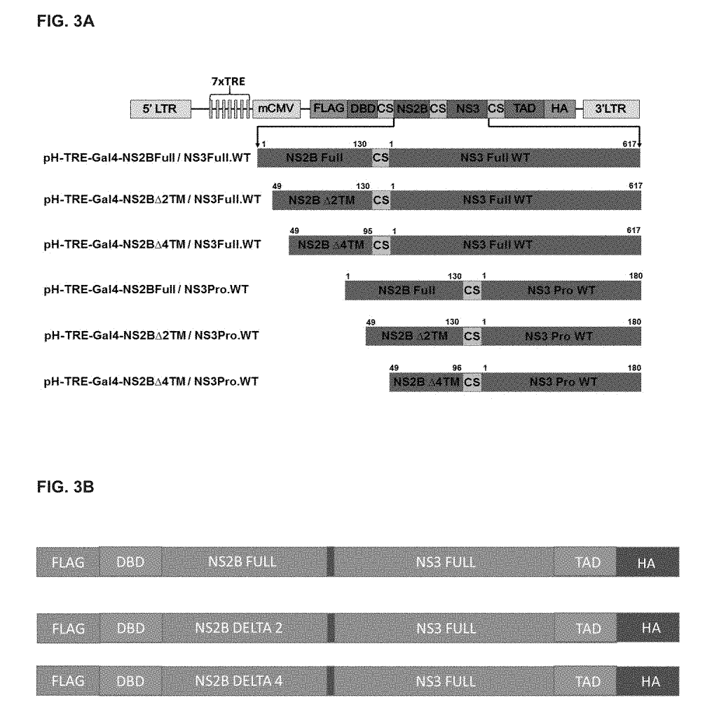

In order to adapt the assay to ZIKV, a set of constructs are engineered. These include first the full NS2B cofactor and NS3 (referred to as ‘Full’ in FIG. 3). We intend to incorporate also the six amino acids upstream of NS2B (at the boundary of NS2A) as well as the amino acids downstream of NS3 (boundary with NS4A). This long protein will be fused in the context of the retroviral plasmid carrying DBD and TAD. As NS2B naturally contains two sets of two hydrophobic transmembrane domains (TMs) that anchor it to the ER, we intend to delete these for the assay to function properly. This was performed already for a DenV assay and was proven feasible; accordingly, these TMs can be effectively removed within NS2B while keeping the protein's function intact.

Deletion of two or four TMs is referred as Δ2TM and Δ4TM in FIG. 3. Constructs will include full-length NS3 or just the catalytic domain (NS3 Pro in FIG. 3). A set of mutant constructs, made by site directed mutagenesis, will replace the Serine at position 135 within the NS3 catalytic triad, which is critical for its activity.

The set of vectors are transfected into mammalian cells for analysis of their phenotype (green or not green as seen by flow cytometry). The best set of wild type and mutant counterpart are chosen to produce cell lines with retroviral technology, as routinely performed in the laboratory. In alternative embodiments, these cell lines are used as a platform for screening.

### Example 2: Exemplary Assays and Constructs

In alternative embodiments, provided are compositions (including exemplary constructs) and assays for screening for Zika Virus (ZIKV) protease, e.g., a ZIKV NS3 protease, inhibitors.

In alternative embodiments, systems and assays as provided herein use Gal4 fusion protein domains, including the DNA binding domain (DBD) and Transactivation domain (TAD), and the viral protease sequence fused between them. The assay then relies on the ability of the fusion protein to activate a fluorescent protein gene such as Green Fluorescent Protein (GFP), used here as an example, which we engineered to be activated by the DBD and TAD domains of Gal4 (when both are present). If protease is active, in the presence of cleavage sites, protease will cleave the fusion and separate the Gal4 domains. As a result, GFP will not be activated and cells will not fluoresce. If a) mutated and inactive, b) in the absence of cleavage sites (used as substrates for protease) or c) when inhibited by inhibitors, the fusion protein will be intact and GFP will be activated. The cells will then fluoresce.

In alternative embodiments, systems and assays as provided herein express a ZIKV protease, comprised of NS2B/NS3 protease sequence (co-factor NS2B and protease NS3). In alternative embodiments, three different related systems of NS2B/NS3 are engineered:
4—In fusion with the Gal4 system for continuous and stable expression;
5—In fusion with the Gal4 system for inducible expression;
6—By themselves, not in fusion with Gal4, for testing their effects on cells, serving also as control in our system.

In order to monitor the activity of the ZIKV NS2B/NS3 protease in the context of this exemplary assay, the following constructs (see below), all based on the cytosolic loop of the NS2B co-factor and the protease domain of NS3, were produced. We constructed four primary constructs that we have utilized to both: demonstrate the feasibility of the assay as well as to produce cell lines aimed at expressing the elements of the assay in a stable manner.

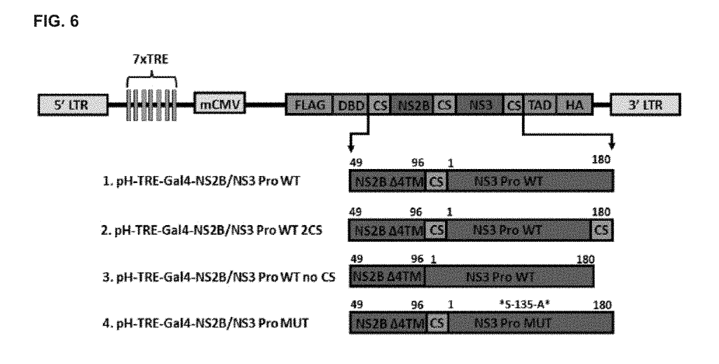

All four constructs contain the hydrophilic portion (the cytosolic loop) of the NS2B cofactor that NS3 requires for catalytic activity of the viral protease. This is the cytosolic loop between the transmembrane motifs of NS2B. As such, the co-factor in the context of the assay lack all its transmembrane domains and is referred to as Δ4TM. These exemplary constructs contain the cytosolic protease-only domain (NS3 Pro) of the NS3 protease that is required and sufficient for cleavage of the NS2B/NS3 cleavage site boundary. The four exemplary constructs, depicted in FIG. 6, are:
1. Wild type NS2B/NS3 with wild-type cleavage NS2B/NS3 boundary (the natural boundary): positive control. This construct should provide an active protease with a substrate. As such it serves as a positive control for the assay and main element for proving the feasibility of the assay for screening or monitoring protease activity. Cells should not fluoresce in its presence.
2. Wild type NS2B/NS3 with wild-type cleavage NS2B/NS3 boundary (the natural boundary) and an additional cleavage site (CS) between NS3 and the TAD domain of Gal4: positive control. We used the NS3/NS4A natural boundary as an additional boundary. This construct has thus two rather than one cleavage boundary (two substrates rather than one). As such it serves as an additional positive control for the assay and main element for proving the feasibility of the assay for screening or monitoring protease activity. Cells should not fluoresce in its presence.
3. Wild type NS2B/NS3 but no cleavage NS2B/NS3 boundary: negative control. This construct should provide an active protease but with no substrate. As such it serves as negative control for the assay. Cells should fluoresce in its presence.
4. Mutant NS2B/NS3 with wild-type cleavage NS2B/NS3 boundary (the natural boundary): negative control. This NS3 Pro MUT construct contains a catalytically inactivated NS3 protease due to a mutation at residue 135 from a serine to an alanine residue within the catalytic triad required for its activity. While this construct has a substrate, it relies on an inactive protease and serves as a true negative control. Cells should fluoresce in its presence.

In alternative embodiments, when mutations at the RNA sequence and/or amino acid sequence level are observed in important or new ZIKV strains, those can be easily incorporated into these exemplary assays as provided herein in such a way that the assay best monitors the activity of the viral protease and its co-factor, as well as providing the best fit cellular platform for drug discovery.

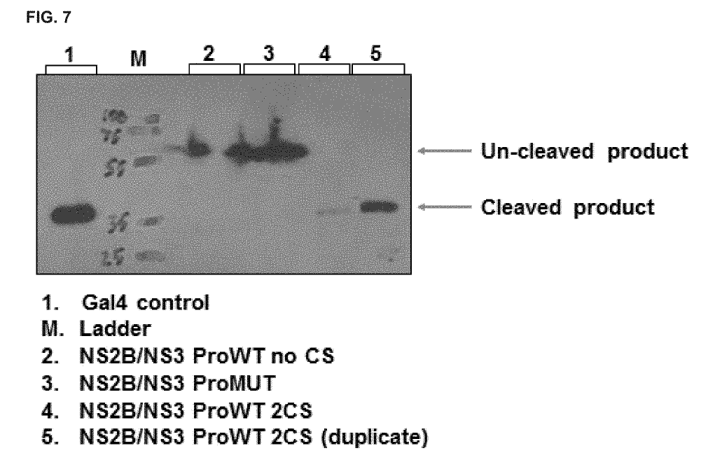

We demonstrated that these constructs carry the protease fusion as described by demonstrating their expression in cells. Experiments detected the fusion protein in cells transduced/infected (lane 2 and 3 in FIG. 7) or transfected (lane 4 and 5 in FIG. 7). Cells were analyzed for Western blot with anti-HA antibody that recognizes the C-terminal fusion downstream the TAD domain of Gal4 (see top of FIG. 6). The Western blot reveals an un-cleaved product with the expected size when the protease is active but does not have a cleavage site (lane 2) or when it is inactive, even in the presence of cleavage site (lane 3). It also reveals an expected cleaved product which should include the TAD domain of Gal4 with the HA tag, but no NS2B co-factor or DNA binding domain (DBD).

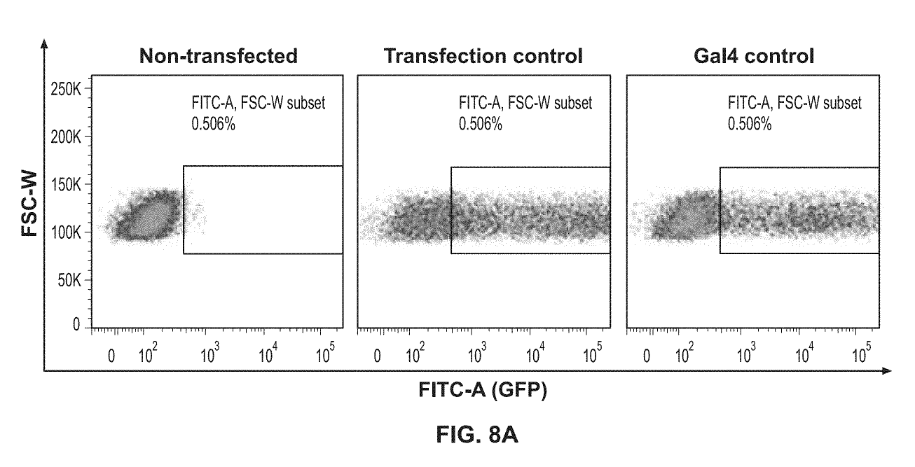

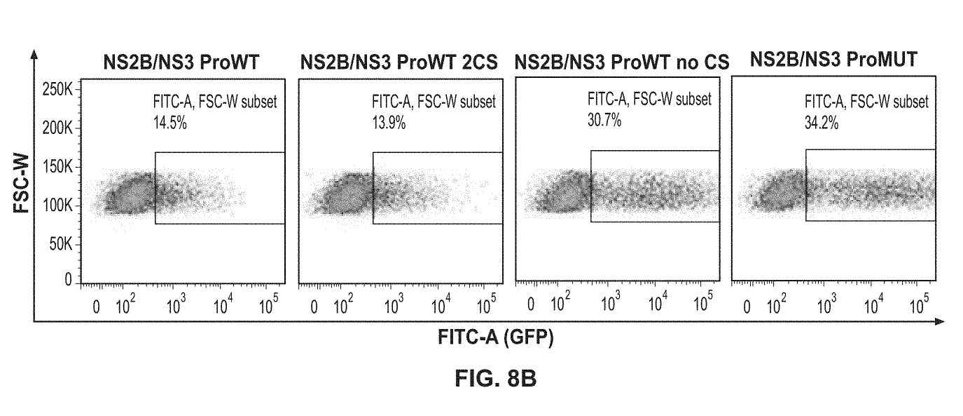

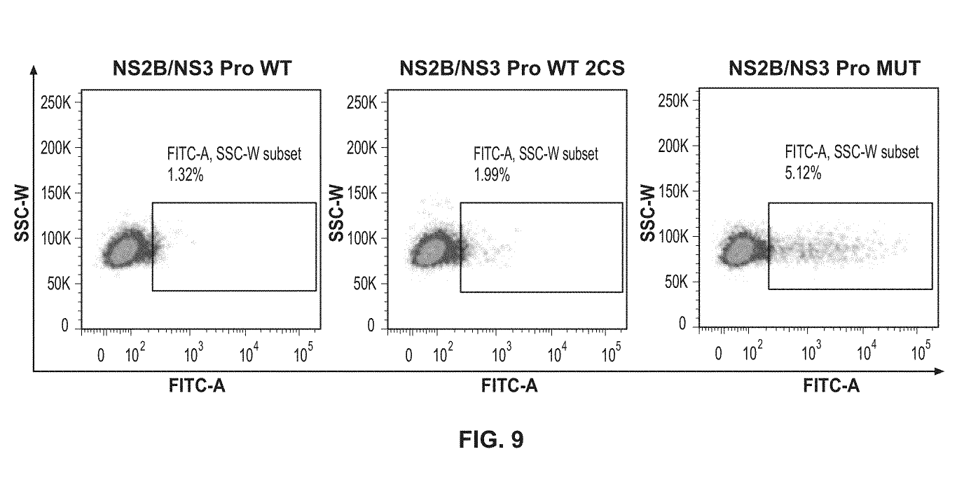

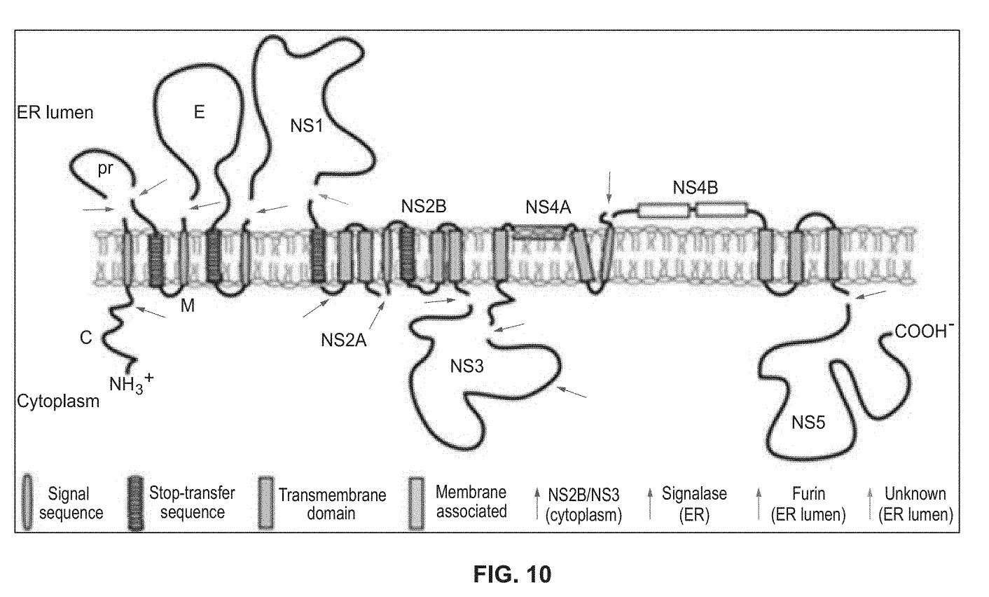

We transfected cells with the different constructs in order to corroborate their effect on GFP activation. Cells were analyzed 48 hours post-transfection. FIG. 8A-B shows the flow cytometry (FACS) analysis of cells transfected with controls (FIG. 8A) and experimental constructs (FIG. 8B). The overall trend is clear: Constructs with active protease (cleaved band in Western blot analysis) show much less green fluorescence than constructs with inactive protease or active but without cleavage site. The difference observed is around 15% versus 32%. This 50% reduction in green fluorescence when active can be further increased. The trend is clearly what the assay is expected to show.

In alternative embodiments, provided are stable cell lines comprising or having contained therein exemplary constructs as provided herein. It was demonstrated that a subpopulation of cells collected post infection (transduction) show a higher degree of fluorescence with the mutant version: 5% versus 1.5 in average. These numbers can be increased.

In conclusion, these transfection experiments, the stable expression assay and the Western blot all corroborate the feasibility of exemplary assays as provided herein for monitoring Zika virus protease activity. As such exemplary assays as provided herein provide an effective cellular platform for drug discovery.

A number of embodiments of the invention have been described. Nevertheless, it will be understood that various modifications may be made without departing from the spirit and scope of the invention. Accordingly, other embodiments are within the scope of the following claims.

What is claimed is:

1. A cell-based method for monitoring the activity of a ZIKV protease or equivalent, comprising:
(a) providing a nucleic acid encoding a chimeric scaffold protein operatively linked to a transcriptional regulatory unit, wherein the chimeric scaffold protein comprises:
(i) an amino acid motif or subsequence susceptible to cleavage by the ZIKV protease or equivalent under physiologic or cell culture conditions;
(ii) a transmembrane domain;
(iii) a signal sequence or any amino acid motif that places the scaffold protein on the extracellular surface of the cell; and
(iv) a detectable moiety,
wherein the amino acid motif or subsequence susceptible to cleavage by the ZIKV protease or equivalent under physiologic or cell culture conditions is positioned within the chimeric scaffold protein such that when the detectable moiety is cleaved away from (off from) the chimeric scaffold protein by the ZIKV protease or equivalent, the remaining subsequence of chimeric scaffold protein on the extracellular surface of the cell lacks the detectable moiety,
and optionally
the ZIKV NS3 proteases have amino acid sequences: ZIKV-2013 (SEQ ID NO:6); ZIKV-2007 (SEQ ID NO:7); ZIKV-1947 (SEQ ID NO:8);
the ZIKV NS2B co-factors have amino acid sequences: ZIKV-2013 (SEQ ID NO:2); ZIKV-2007 (SEQ ID NO:3); ZIKV-1947 (SEQ ID NO:4);
(b) providing a nucleic acid encoding the ZIKV protease or equivalent operatively linked to a transcriptional regulatory unit, or a cell that expresses a heterologous or endogenous form of the ZIKV protease or equivalent;
(c) inserting (transfecting) the nucleic acid of (a) and (b) into the cell if the cell does not already express a heterologous or endogenous form of the ZIKV protease or equivalent protease;
(d) co-expressing the nucleic acid of (a) and (b) in the cell, or expressing the nucleic acid in the cell if the cell already expresses a heterologous or endogenous form of the ZIKV protease or equivalent protease; and
(e) determining whether the chimeric scaffold protein comprising the detectable moiety is expressed on the extracellular surface of the cell,
wherein an intact scaffold protein comprising the detectable moiety is expressed on the extracellular surface of the cell when the ZIKV protease or equivalent protease is not enzymatically active, and an intact chimeric scaffold protein is not or is substantially less expressed on the extracellular surface of the cell when the ZIKV protease or equivalent protease is enzymatically active.

2. An isolated, chimeric, recombinant or synthetic nucleic acid encoding a chimeric scaffold protein,
wherein the nucleic acid is operatively linked to a transcriptional regulatory unit,
and wherein the chimeric scaffold protein comprises:
(1) (a) (i) an amino acid motif or subsequence susceptible to cleavage by a ZIKV protease or equivalent protease under physiologic or cell culture conditions,
and optionally
the ZIKV NS3 proteases have amino acid sequences: ZIKV-2013 (SEQ ID NO:6); ZIKV-2007 (SEQ ID NO:7); ZIKV-1947 (SEQ ID NO:8);
(ii) a transmembrane domain;
(iii) a signal sequence or any amino acid motif that places the chimeric scaffold protein on the extracellular surface of the cell; and
(iv) a detectable moiety;
wherein the chimeric scaffold protein comprises an endoplasmic reticulum (ER) retention motif or a KDEL (SEQ ID NO:1) motif,
wherein the ER retention motif or KDEL (SEQ ID NO:1) motif is positioned in the chimeric scaffold protein such that when the ZIKV protease or equivalent protease is active the chimeric scaffold will be separated into two pieces, leaving the ER retention motif-comprising or KDEL (SEQ ID NO:1) motif-comprising portion of the polypeptide in the ER and freeing the detectable moiety-comprising portion to the cell's extracellular membrane, and if the ZIKV protease or equivalent protease is blocked or inactive, the entire chimeric scaffold polypeptide will be retained in the ER, and as a consequence will not be detected on the cell's extracellular surface.

3. A chimeric polypeptide encoded by the isolated, recombinant or synthetic nucleic acid of claim 2.

4. The cell-based method of claim 1, further comprising screening for an inhibitor of ZIKV protease or equivalent by:
(a) providing a compound to be screened as an inhibitor of the ZIKV protease or equivalent enzyme, or providing a nucleic acid to be screened as encoding an inhibitor of the enzyme;
(b) contacting a plurality of the cells with the compound or nucleic acid either before, during and/or after the co-expressing the nucleic acid in the cell; and
(c) determining whether the chimeric scaffold protein comprising the detectable moiety is expressed on the extracellular surface of the cell,
wherein an intact chimeric scaffold protein comprising the detectable moiety is expressed on the extracellular surface of the cell when the ZIKV protease or equivalent enzyme, is inhibited by: the compound, a composition encoded by the nucleic acid, or a compound present in the cell only because the nucleic acid was expressed, and an intact chimeric scaffold protein is not or is substantially less expressed on the extracellular surface of the cell the ZIKV protease or equivalent enzyme, is
enzymatically active, and the enzymatic activity of the ZIKV protease or equivalent enzyme, is not significantly inhibited by: the compound, a composition encoded by the nucleic acid, or a compound present in the cell only because the nucleic acid was expressed.

5. The cell-based method of claim 1, further comprising running a negative control comprising dividing the plurality of the cells co-expressing the nucleic acid of (a) and (b) in the cell and not adding the compound to be screened as an inhibitor to one of the divided cell samples.

6. The cell-based method of claim 1, further comprising running a positive control comprising dividing the plurality of the cells co-expressing the nucleic acid of (a) and (b) in the cell and adding a known inhibitor of the enzyme, to one of the divided cell samples.

7. The cell-based method of claim 1, wherein the transcriptional regulatory unit comprises a promoter, an inducible promoter or a constitutive promoter.

8. The cell-based method of claim 1, wherein the cell is a mammalian cell, a monkey cell or a human cell, or a lymphocyte or a hepatocyte, or a T cell; and optionally the cells are genetically bar-coded.

9. The cell-based method of claim 1, wherein the chimeric scaffold protein comprises all or part of a mouse Lyt2 or a human CD8 polypeptide.

10. The cell-based method of claim 1, wherein the wherein the detectable moiety comprises an epitope for an antibody, or a FLAG tag.

11. The cell-based method of claim 1, wherein the detectable moiety is detected or measured on the extracellular surface of the cell by a high throughput screen, a plate-reader, a flow cytometry or microscope visualization.

12. The cell-based method of claim 1, wherein the compound to be screened as an inhibitor of the enzyme: comprises a small molecule, a nucleic acid, a polypeptide or peptide, a peptidomimetic, a polysaccharide or a lipid; is a member of a library of compounds to be screened, or is a member of a random peptide library or a chemical compound library.

13. The cell-based method of claim 1, wherein the two or more, or a plurality of, enzymes are screened in the same cell; and, wherein optionally the enzyme or enzymes are variants of the same enzyme or a different enzyme or a combination thereof.

14. The isolated, chimeric, recombinant or synthetic nucleic acid of claim 2, wherein the scaffold protein comprises all or part of a mouse Lyt2 or a human CD8 polypeptide.

15. The isolated, chimeric, recombinant or synthetic nucleic acid of claim 2, wherein the detectable moiety comprises an epitope for an antibody, or a FLAG tag.

16. A cell-based method for monitoring the activity of a ZIKV protease or equivalent, comprising:
(a) providing a first chimeric nucleic acid encoding a chimeric protein, wherein the nucleic acid is operatively linked to a constitutive or an inducible transcriptional activator (optionally a constitutive or an inducible promoter),
where the chimeric protein comprises a proteolytically active ZIKV protease or equivalent and its cofactor polypeptide NS2B, wherein the NS2B polypeptide lacks its transmembrane domain (TM), and the proteolytically active ZIKV protease or equivalent is capable of recognizing and cleaving a specific cleavage site (CS), and the CS is positioned between the NS2B polypeptide and the proteolytically active ZIKV protease or equivalent,
and the proteolytically active ZIKV protease or equivalent and its cofactor polypeptide NS2B is positioned within the chimeric protein between two domains of a transcription factor comprising a DNA-binding domain (DBD) and a C-terminal Transactivation domain (TAD), wherein optionally the DBD and the TAD are derived from a yeast Gal4 protein transcription factor, and the transcriptional factor is active only if the DBD and the TAD are on or contained within the same chimeric protein,
and optionally the first nucleic acid comprises a construct as illustrated in FIG. 3 or FIG. 6, optionally a construct comprising: pH-TRE-Gal4-NS2B/NS3 Protease (pro) WT (wild type); or, pH-TRE-Gal4-NS2B/NS3 Pro WT 2CS (cleavage site),
and optionally the ZIKV NS3 protease has an amino acid sequence: ZIKV-2013 (SEQ ID NO:6); ZIKV-2007 (SEQ ID NO:7); ZIKV-1947 (SEQ ID NO:8);
and optionally the ZIKV NS3 Virus Polyprotein has a sequence comprising: NIBR Accession Number: AAV34151.1; NIBR Accession Number: ACD75819.1; or NIBR Accession Number: AHZ13508.1,
and optionally the ZIKV NS2B co-factors have amino acid sequences: ZIKV-2013 (SEQ ID NO:2); ZIKV-2007 (SEQ ID NO:3); ZIKV-1947 (SEQ ID NO:4);
and optionally the cleavage sites (CS) between NS2B/NS3, and NS3/NS4A for: ZIKV-2013=NS2B/NS3 (SEQ ID NO:9) and NS3/NS4A (SEQ ID NO:10); ZIKV-2007=NS2B/NS3 (SEQ ID NO:11) and NS3/NS4A (SEQ ID NO:12); ZIKV-1947=NS2B/NS3 (SEQ ID NO:13) and NS3/NS4A (SEQ ID NO:14); and/or (Dengue virus) DENV-2=NS2B/NS3 (SEQ ID NO:15) and NS3/NS4A (SEQ ID NO:16), where the “/” indicates the site of cleavage,
and optionally the cleavage site (CS) is a Zika Virus Polyprotein Cleavage Sites comprise:
Capsid C-Terminus Hydrophobic//Pre-membrane

| | |
|---|---|
| | (SEQ ID NO: 17) |
| MR-766 | 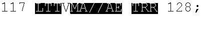 |
| | (SEQ ID NO: 18) |
| Yap-2007 | 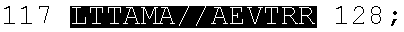 |
| | or |
| | (SEQ ID NO: 19) |
| FP-2013 | 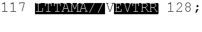 |

Pre-membrane//Membrane

| | |
|---|---|
| | (SEQ ID NO: 20) |
| MR-766 | 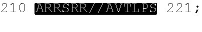 |
| | (SEQ ID NO: 21) |
| Yap-2007 | 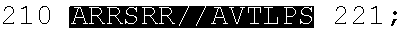 |
| | or |
| | (SEQ ID NO: 22) |
| FP-2013 | 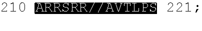 |

Membrane//Envelope

| | |
|---|---|
| | (SEQ ID NO: 23) |
| MR-766 | 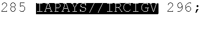 |
| | (SEQ ID NO: 24) |
| Yap-2007 | 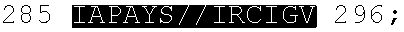 |
| | or |
| | (SEQ ID NO: 25) |
| FP-2013 | 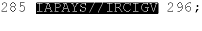 |

Envelope/NS1

| | |
|---|---|
| | (SEQ ID NO: 26) |
| MR-766 | 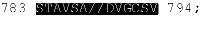 |
| | (SEQ ID NO: 27) |
| Yap-2007 | 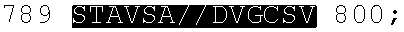 |
| | or |
| | (SEQ ID NO: 28) |
| FP-2013 | 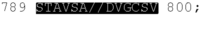 |

NS1//NS2A

| | |
|---|---|
| | (SEQ ID NO: 29) |
| MR-766 | 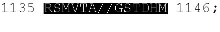 |
| | (SEQ ID NO: 30) |
| Yap-2007 | 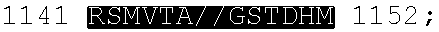 |
| | or |
| | (SEQ ID NO: 31) |
| FP-2013 | 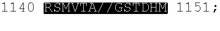 |

NS2A//NS2B

| | |
|---|---|
| | (SEQ ID NO: 32) |
| MR-766 | 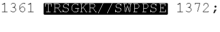 |
| | (SEQ ID NO: 33) |
| Yap-2007 | 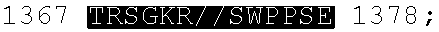 |
| | or |
| | (SEQ ID NO: 34) |
| FP-2013 | 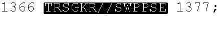 |

NS2B//NS3

| | |
|---|---|
| | (SEQ ID NO: 35) |
| MR-766 | 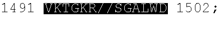 |
| | (SEQ ID NO: 36) |
| Yap-2007 | 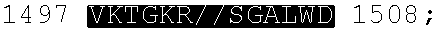 |
| | or |
| | (SEQ ID NO: 37) |
| FP-2013 | 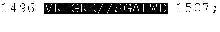 |

NS3//NS4A

| | |
|---|---|
| | (SEQ ID NO: 38) |
| MR-766 | 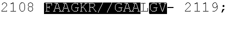 |
| | (SEQ ID NO: 39) |
| Yap-2007 | 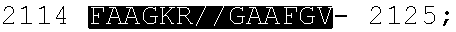 |
| | or |
| | (SEQ ID NO: 40) |
| FP-2013 | 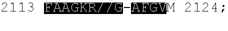 |

NS4A//2K

| | |
|---|---|
| | (SEQ ID NO: 41) |
| MR-766 | 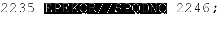 |
| | (SEQ ID NO: 42) |
| Yap-2007 | 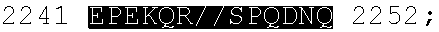 |
| | or |
| | (SEQ ID NO: 43) |
| FP-2013 | 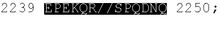 |

2K//NS4B

| | |
|---|---|
| | (SEQ ID NO: 44) |
| MR-766 | 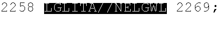 |
| | (SEQ ID NO: 45) |
| Yap-2007 | 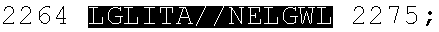 |
| | or |
| | (SEQ ID NO: 46) |
| FP-2013 | 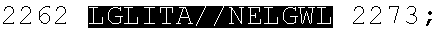 |
| | or |

NS4B//NS5

| | |
|---|---|
| | (SEQ ID NO: 47) |
| MR-766 | 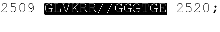 |
| | (SEQ ID NO: 48) |
| Yap-2007 | 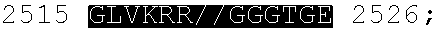 |
| | or |
| | (SEQ ID NO: 49) |
| FP-2013 | 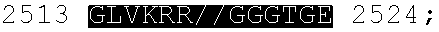 |

(b) providing a second chimeric nucleic acid encoding a detectable moiety or a detectable protein, wherein the nucleic acid is operatively linked to a promoter activated by the transcription factor encoded by the first chimeric nucleic acid of (a),
and optionally the detectable moiety or the detectable protein is or comprises a fluorescent protein or a luminescent protein, and optionally the fluorescent protein is a Green Fluorescent Protein (GFP), an enhanced Green Fluorescent Protein (eGFP), or a luciferase,
and optionally the second chimeric nucleic acid is contained in a same or a different construct as the first chimeric nucleic acid, wherein optionally the construct is a plasmid, a viral vector or a retroviral vector,
(c) inserting or transfecting the first chimeric nucleic acid of (a) and the second chimeric nucleic acid of (b) into a cell, wherein if the first chimeric nucleic acid of (a) and the second chimeric nucleic acid of (b) are on the same construct, then inserting or transfecting the construct into the cell,
wherein optionally the cell is genetically bar-coded;
(d) co-expressing the first chimeric nucleic acid of (a) and the second chimeric nucleic acid of (b) in the cell; and
(e) determining whether the detectable moiety or the detectable protein is expressed in the cell,
wherein if the ZIKV protease or equivalent is active it will cleave the chimeric protein at the cleavage site or cleavage sites and separate the DBD and the TAD, and as a result the transcription factor, lacking a DBD and an TAD on the same chimeric protein, cannot bind to and activate the promoter on the second chimeric nucleic acid to result in transcription of the detectable moiety or the detectable protein, and thus the cells will not express the detectable moiety or the detectable protein, and if the detectable moiety or the detectable protein is a fluorescent or a luminescent protein, the cell will not fluoresce or luminesce,
and if the ZIKV protease or equivalent is inactive or inactivated, the cells will express the detectable moiety or the detectable protein, and if the detectable moiety or the detectable protein is a fluorescent or a luminescent protein, the cell will fluoresce or luminesce.

17. A cell-based method for screening of an activator or inhibitor of a ZIKV protease or equivalent, comprising: the method of claim 16, further comprising exposing the cell, or adding to the cell, or expressing in the cell, a potential, putative or candidate inhibitor or activator of the ZIKV protease or equivalent,
wherein if the ZIKV protease or equivalent is inactivated by the potential, putative or candidate inhibitor, the cells will express the detectable moiety or the detectable protein, and if the detectable moiety or the detectable protein is a fluorescent or a luminescent protein, the cell will fluoresce or luminesce.

18. The cell-based method of claim 17, wherein the potential, putative or candidate inhibitor or activator of the ZIKV protease or equivalent to be screened comprises: a small molecule, a nucleic acid, a polypeptide or peptide, a peptidomimetic, a polysaccharide or a lipid; is a member of a library of compounds to be screened, or is a member of a random peptide library or a chemical compound library.

19. The cell-based method of claim 17, further comprising:
(a) running a negative control comprising dividing a plurality of the cells co-expressing the first chimeric nucleic acid of (a) and the second chimeric nucleic acid of (b) in the cell and not adding the potential, putative or candidate inhibitor or activator of the ZIKV protease or equivalent to be screened to one of the divided cell samples; or
(b) running a positive control comprising dividing the plurality of the cells co-expressing the first chimeric nucleic acid of (a) and the second chimeric nucleic acid of (b) in the cell and adding a known inhibitor of the ZIKV protease or equivalent to one of the divided cell samples.

20. A recombinant or engineered isolated cell comprising the first chimeric nucleic acid or the first chimeric nucleic acid and the second chimeric nucleic acid used in the method of claim 16.

21. A construct comprising or having contained therein a nucleic acid encoding the first chimeric nucleic acid or the first chimeric nucleic acid and the second chimeric nucleic acid used in the method of claim 16, wherein optionally the construct is a plasmid, a vector or a recombinant vector.


SEQUENCE LISTING (49 sequences)

SEQ ID NO: 1 - protein, 4 aa, Artificial Sequence
  Synthetic peptide motif
KDEL

SEQ ID NO: 2 - protein, 142 aa, Zika virus
  misc_feature (1)..(142): ZIKV-2013 (French Polynesia Strain)
TRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWD

SEQ ID NO: 3 - protein, 142 aa, Zika virus; identical to SEQ ID NO: 2
  misc_feature (1)..(142): ZIKV-2007 (Micronesia Strain)

SEQ ID NO: 4 - protein, 142 aa, Zika virus
  misc_feature (1)..(142): ZIKV-1947 (YAP Ethiopia strain)
TRSGKRSWPPSEVLTAVGLICALAGGFAKADIEMAGPMAAVGLLIVSYVVSGKSVDMYIERAGDITWEKDAEVTGNSPRLDVALDESGDFSLVEEDGPPMREIILKVVLMAICGMNPIAIPFAAGAWYVYVKTGKRSGALWD

SEQ ID NO: 5 - protein, 142 aa, Dengue virus 2
  misc_feature (1)..(142): (Dengue virus) DENV-2
RTNKKRSWPLNEAIMAVGMVSILASSLLKNDIPMTGPLVAGGLLTVCYVLTGRSADLELERAADVRWEEQAEISGSSPILSITISEDGSMSIKNEEEEQTLTILIRTGLLVVSGLFPVSTPITAAAWYLWEVKKQRAGVLWD

SEQ ID NO: 6 - protein, 629 aa, Zika virus
  misc_feature (1)..(629): ZIKV-2013 (French Polynesia Strain)
VKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGV

SEQ ID NO: 7 - protein, 628 aa, Zika virus
  misc_feature (1)..(628): ZIKV-2007 (Micronesia Strain)
VKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTPPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFLKTKNQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRKWCFDGTTNNTIMEDSVPAEVWTRYGEKRVLKPRWMDARVCSDHAALKSFKEFAAGKRGAFGV

SEQ ID NO: 8 - protein, 629 aa, Zika virus
  misc_feature (1)..(629): ZIKV-1947 (YAP Ethiopia strain)
VKTGKRSGALWDVPAPKEVKKGETTDGVYRVMTRRLLGSTQVGVGVMQEGVFHTMWHVTKGAALRSGEGRLDPYWGDVKQDLVSYCGPWKLDAAWDGLSEVQLLAVPPGERARNIQTLPGIFKTKDGDIGAVALDYPAGTSGSPILDKCGRVIGLYGNGVVIKNGSYVSAITQGKREEETPVECFEPSMLKKKQLTVLDLHPGAGKTRRVLPEIVREAIKKRLRTVILAPTRVVAAEMEEALRGLPVRYMTTAVNVTHSGTEIVDLMCHATFTSRLLQPIRVPNYNLNIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKNQEWDFVITTDISEMGANFKADRVIDSRRCLKPVILDGERVILAGPMPVTHASAAQRRGRIGRNPNKPGDEYMYGGGCAETDEGHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTKYGEKRVLKPRWMDARVCSDHAALKSFKEFAAGKRGAALGV

SEQ ID NO: 9 - protein, 12 aa, Zika virus
  misc_feature (1)..(12): ZIKV-2013 NS2B/NS3
VKTGKRSGALWD

SEQ ID NO: 10 - protein, 12 aa, Zika virus
  misc_feature (1)..(12): ZIKV-2013 NS3/NS4A
FAAGKRGAAFGV

SEQ ID NO: 11 - protein, 12 aa, Zika virus
  misc_feature (1)..(12): ZIKV-2007 NS2B/NS3
VKTGKRSGALWD

SEQ ID NO: 12 - protein, 11 aa, Zika virus
  misc_feature (1)..(11): ZIKV-2007 NS3/NS4A
FAAGKRGAFGV

SEQ ID NO: 13 - protein, 12 aa, Zika virus
  misc_feature (1)..(12): ZIKV-1947 NS2B/NS3
VKTGKRSGALWD

SEQ ID NO: 14 - protein, 12 aa, Zika virus
  misc_feature (1)..(12): ZIKV-1947 NS3/NS4A
FAAGKRGAALGV

SEQ ID NO: 15 - protein, 12 aa, Dengue virus 2
  misc_feature (1)..(12): DENV-2 NS2B/NS3
EVKKQRAGVLWD

SEQ ID NO: 16 - protein, 12 aa, Dengue virus 2
  misc_feature (1)..(12): DENV-2 NS3/NS4A
FAAGRKSLTLNL

SEQ ID NO: 17 - protein, 12 aa, Zika virus
  misc_feature (1)..(12): Capsid C-Terminus Hydrophobic//Pre-membrane MR-766
LTTVMAAEITRR

SEQ ID NO: 18 - protein, 12 aa, Zika virus
  misc_feature (1)..(12): Capsid C-Terminus Hydrophobic//Pre-membrane Yap-2007
LTTAMAAEVTRR

SEQ ID NO: 19 - protein, 12 aa, Zika virus
  misc_feature (1)..(12): Capsid C-Terminus Hydrophobic//Pre-membrane FP-2013
LTTAMAVEVTRR

SEQ ID NO: 20 - protein, 12 aa, Zika virus
  misc_feature (1)..(12): Pre-membrane//Membrane MR-766
ARRSRRAVTLPS

SEQ ID NO: 21 - protein, 12 aa, Zika virus
  misc_feature (1)..(12): Pre-membrane//Membrane Yap-2007
ARRSRRAVTLPS

SEQ ID NO: 22 - protein, 12 aa, Zika virus
  misc_feature (1)..(12): Pre-membrane//Membrane FP-2013
ARRSRRAVTLPS

SEQ ID NO: 23 - protein, 12 aa, Zika virus
  misc_feature (1)..(12): Membrane//Envelope MR-766
IAPAYSIRCIGV

SEQ ID NO: 24 - protein, 12 aa, Zika virus
  misc_feature (1)..(12): Membrane//Envelope Yap-2007
IAPAYSIRCIGV

SEQ ID NO: 25 - protein, 12 aa, Zika virus
  misc_feature (1)..(12): Membrane//Envelope FP-2013
IAPAYSIRCIGV

SEQ ID NO: 26 - protein, 12 aa, Zika virus
  misc_feature (1)..(12): Envelope//NS1 MR-766
STAVSADVGCSV

SEQ ID NO: 27 - protein, 12 aa, Zika virus
  misc_feature (1)..(12): Envelope//NS1 Yap-2007
STAVSADVGCSV

SEQ ID NO: 28 - protein, 12 aa, Zika virus
  misc_feature (1)..(12): Envelope//NS1 FP-2013
STAVSADVGCSV

SEQ ID NO: 29 - protein, 12 aa, Zika virus
  misc_feature (1)..(12): NS1//NS2A MR-766
RSMVTAGSTDHM

SEQ ID NO: 30 - protein, 12 aa, Zika virus
  misc_feature (1)..(12): NS1//NS2A Yap-2007
RSMVTAGSTDHM

SEQ ID NO: 31 - protein, 12 aa, Zika virus
  misc_feature (1)..(12): NS1//NS2A FP-2013
RSMVTAGSTDHM

SEQ ID NO: 32 - protein, 12 aa, Zika virus
  misc_feature (1)..(12): NS2A//NS2B MR-766
TRSGKRSWPPSE

SEQ ID NO: 33 - protein, 12 aa, Zika virus
  misc_feature (1)..(12): NS2A//NS2B Yap-2007
TRSGKRSWPPSE

SEQ ID NO: 34 - protein, 12 aa, Zika virus
  misc_feature (1)..(12): NS2A//NS2B FP-2013
TRSGKRSWPPSE

SEQ ID NO: 35 - protein, 12 aa, Zika virus
  misc_feature (1)..(12): NS2B//NS3 MR-766
VKTGKRSGALWD

SEQ ID NO: 36 - protein, 12 aa, Zika virus
  misc_feature (1)..(12): NS2B//NS3 Yap-2007
VKTGKRSGALWD

SEQ ID NO: 37 - protein, 12 aa, Zika virus
  misc_feature (1)..(12): NS2B//NS3 FP-2013
VKTGKRSGALWD

SEQ ID NO: 38 - protein, 12 aa, Zika virus
  misc_feature (1)..(12): NS3//NS4A MR-766
FAAGKRGAALGV

SEQ ID NO: 39 - protein, 12 aa, Zika virus
  misc_feature (1)..(12): NS3//NS4A Yap-2007
FAAGKRGAAFGV

SEQ ID NO: 40 - protein, 12 aa, Zika virus
  misc_feature (1)..(12): NS3//NS4A FP-2013
FAAGKRGAFGVM

SEQ ID NO: 41 - protein, 12 aa, Zika virus
  misc_feature (1)..(12): NS4A//2K MR-766
EPEKQRSPQDNQ

SEQ ID NO: 42 - protein, 12 aa, Zika virus
  misc_feature (1)..(12): NS4A//2K Yap-2007
EPEKQRSPQDNQ

SEQ ID NO: 43 - protein, 12 aa, Zika virus
  misc_feature (1)..(12): NS4A//2K FP-2013
EPEKQRSPQDNQ

SEQ ID NO: 44 - protein, 12 aa, Zika virus
  misc_feature (1)..(12): 2K//NS4B MR-766
LGLITANELGWL

SEQ ID NO: 45 - protein, 12 aa, Zika virus
  misc_feature (1)..(12): 2K//NS4B Yap-2007
LGLITANELGWL

SEQ ID NO: 46 - protein, 12 aa, Zika virus
  misc_feature (1)..(12): 2K//NS4B FP-2013
LGLITANELGWL

SEQ ID NO: 47 - protein, 12 aa, Zika virus
  misc_feature (1)..(12): NS4B//NS5 MR-766
GLVKRRGGGTGE

SEQ ID NO: 48 - protein, 12 aa, Zika virus
  misc_feature (1)..(12): NS4B//NS5 Yap-2007
GLVKRRGGGTGE

SEQ ID NO: 49 - protein, 12 aa, Zika virus
  misc_feature (1)..(12): NS4B//NS5 FP-2013
GLVKRRGGGTGE